(19) United States
(12) Patent Application Publication
(10) Pub. No.: US 2024/0350759 A1
(43) Pub. Date: Oct. 24, 2024

(54) METHOD AND SYSTEM OF MONITORING OXYGEN

(71) Applicant: Fisher & Paykel Healthcare Limited, Auckland (NZ)

(72) Inventors: Cameron Leslie MATTHEWS, Auckland (NZ); Rachael GLAVES, Auckland (NZ); Lindy Talia LOWENSTEIN, Auckland (NZ); Gareth Thomas MCDERMOTT, Auckland (NZ)

(21) Appl. No.: 18/685,657

(22) PCT Filed: Aug. 25, 2022
(86) PCT No.: PCT/IB2022/057948

§ 371 Date: Feb. 22, 2024

Related U.S. Application Data
(60) Provisional application No. 63/260,649, filed on Aug. 27, 2021.

(51) Int. Cl. A61M 16/00; A61M 16/06; A61M 16/10

(52) U.S. Cl. CPC A61M 16/024; A61M 16/0003; A61M 16/0051; A61M 16/0066; A61M 16/0672; A61M 16/109; A61M 16/1095; A61M 2202/0007; A61M 2202/0208; A61M 2205/10; A61M 2205/18; A61M 2205/3327; A61M 2205/36; A61M 2205/502; A61M 2205/581; A61M 2205/583; A61M 2230/40

(57) ABSTRACT

The invention is directed to a method of monitoring oxygen at a patient to determine spontaneous breathing with a patent airway. The method includes the steps of providing a gas flow to a patient's airway, the gas flow including a predetermined fraction of oxygen, monitoring the fraction of oxygen at the patient's airway, generating a waveform representing the fraction of oxygen at the patient's airway, and determining whether the patient is spontaneously breathing with a patent airway based on the waveform.

### TECHNICAL FIELD

The present invention relates to a method and system of monitoring oxygen to determine spontaneous breathing with a patent airway.

### BACKGROUND OF INVENTION

When a patient is receiving respiratory support, it is often beneficial to monitor gases at the patient's airway to provide useful feedback to clinicians. Patient monitoring can be particularly useful when the patient has diminished respiratory function or a risk of diminished respiratory function, for example during a medical procedure when anaesthetic agents are used. Patient monitoring can for example detect when a spontaneously breathing patient becomes apnoeic or is experiencing an obstructed airway, and alert a clinician, preferably before the patient's blood oxygen level deteriorates to a dangerous level and/or the patient's carbon dioxide level rises to a dangerous level.

It is known to monitor carbon dioxide levels at the patient to determine whether the patient is spontaneously breathing with a patent airway. However, monitoring carbon dioxide levels at a patient becomes difficult when a gas flow is provided to the patient because the gas flow can dilute the carbon dioxide levels.

Embodiments of the invention may provide a method and system of monitoring oxygen to determine spontaneous breathing with a patent airway, which overcomes or ameliorates one or more of the disadvantages or problems described above, or which at least provides the consumer with a useful choice.

A reference herein to a patent document or any other matter identified as prior art, is not to be taken as an admission that the document or other matter was known or that the information it contains was part of the common general knowledge as at the priority date of any of the claims.

### SUMMARY OF INVENTION

According to one aspect of the invention, there is provided a method of monitoring oxygen at a patient to determine spontaneous breathing with a patent airway, the method including the steps of providing a gas flow to a patient's airway, the gas flow including a predetermined fraction of oxygen, monitoring the fraction of oxygen at the patient's airway, generating a waveform representing the fraction of oxygen at the patient's airway, and determining whether the patient is spontaneously breathing with a patent airway based on the waveform.

In this specification, the terms “oxygen fraction”, “fraction of oxygen” and “oxygen concentration” may be used interchangeably. The oxygen concentration of ambient air is an oxygen fraction of 21% (this can be expressed as 0.21). In another example, pure oxygen has an oxygen fraction of 100% (this can be expressed as 1).

In one embodiment, the patient's airway includes the patient's nose. In another embodiment, the patient's airway includes the patient's mouth. Optionally, the patient's airway may include any one or both of the patient's nose and mouth. The step of monitoring may include monitoring the fraction of oxygen inside the patient's nasal passage, or outside/proximate the patient's nasal passage. The step of monitoring may include monitoring the fraction of oxygen inside the patient's mouth, or outside/proximate the patient's mouth. Accordingly, throughout the specification, references to monitoring the fraction of oxygen at the patient's airway may refer to monitoring the fraction of oxygen inside/outside/proximate the patient's nose and/or inside/outside/proximate the patient's mouth.

Typically, the gas flow is provided via a gas delivery apparatus. The gas delivery apparatus may include a flow source for providing the gas flow. The flow source may include an oxygen source for providing oxygen in the gas flow. The flow source may further include a flow generator for controlling the flow rate of the gas flow.

The step of monitoring the fraction of oxygen at the patient's airway may include using one or more sensors to monitor the fraction of oxygen at the patient's airway. In one example, the one or more sensors may be positioned within and/or proximate the patient's airway for directly measuring the fraction of oxygen at the patient's airway. In another example, the one or more sensors may be positioned remotely from the patient. In this example, a gas sampling interface may be used to sample gases at the patient's airway and the sampled gases may be delivered to the one or more remote sensors for measuring the fraction of oxygen. In particular, the one or more sensors may include an oxygen sensor.

The step of generating a waveform may include using a controller to generate the waveform based on input from the one or more sensors. The controller may generate the waveform for display on an electronic display device.

The waveform may represent the fraction of oxygen at the patient's airway over time. Typically, when the patient is breathing with a patent airway, the waveform indicates changes in the fraction of oxygen for each respiratory cycle as measured at the patient's airway. Conversely, a waveform that illustrates insufficient changes in the fraction of oxygen for each assumed respiratory cycle or inconsistencies between assumed respiratory cycles may indicate diminished respiratory function or a risk of diminished respiratory function, that the patient is apnoeic, and/or partial or full obstruction of the patient's airway.

In one embodiment, the step of determining includes determining that the patient is spontaneously breathing with a patent airway when the waveform indicates a decrease in the fraction of oxygen with respect to a baseline fraction of oxygen delivered to the patient corresponding to an assumed respiratory cycle of the patient.

In practice, the fraction of oxygen provided in the gas flow by the flow source can be different to the fraction of oxygen delivered to the patient at the patient's airway. In particular, the fraction of oxygen delivered to the patient at the patient's airway may be less than the fraction of oxygen provided in the gas flow from the flow source, for example due to entrainment of gases at the patient interface. In scenarios where the patient is fitted with a non-sealing patient interface, an entrainment of gases can dilute the fraction of oxygen provided by the flow source, resulting in a lower delivered fraction of oxygen to the patient. Entrainment of gases may occur, for example, as a result of the provided flow rate not meeting the patient's inspiratory demand, and/or the use of a non-sealing patient interface.

In one embodiment, the step of determining includes determining that the patient is apnoeic or has an obstructed airway when the waveform indicates insufficient change in the fraction of oxygen over a period of time as measured at the patient's airway. In particular, the method may include automatically detecting that the patient may be apnoeic or may have an obstructed airway based on the waveform. The step of automatic detection may be carried out by a controller. In particular, the controller may compare the fraction of oxygen at the patient's airway against predetermined thresholds, and automatically make the detection if the monitored change in the fraction of oxygen falls outside the predetermined thresholds. In one example, it may be determined that the patient is apnoeic and/or has an obstructed airway if there is 0% change or near 0% change in the fraction of oxygen at the patient's airway over a period of time. In another example, it may be determined that the patient has a partially obstructed airway if there is a notable decrease in the percentage change in the fraction of oxygen at the patient's airway over a period of time.

Optionally, the step of providing a gas flow to the patient's airway includes providing the gas flow through a non-sealing user interface. The non-sealing user interface may include a non-sealing nasal cannula. In some alternative embodiments, the step of providing a gas flow to the patient's airway may include providing the gas flow through a sealing user interface.

The step of determining may include determining that the patient is spontaneously breathing with a patent airway if the waveform indicates a change in oxygen fraction with respect to a baseline oxygen fraction delivered to the patient of more than about 10%. In particular, the step of determining may include determining that the patient is spontaneously breathing with a patent airway if the waveform indicates a change in oxygen fraction with respect to a baseline oxygen fraction delivered to the patient of more than about 30%.

The method may further include determining that the patient is spontaneously breathing with a patent airway, if the waveform indicates at least two dips during a period of assumed respiratory cycle when an inspiratory demand of the patient is not met.

The method may further include determining that the patient is spontaneously breathing with a patent airway if the waveform indicates at least two dips during a period of assumed respiratory cycle when an inspiratory demand of the patient is not met, and the waveform indicates a change in oxygen fraction with respect to a baseline oxygen fraction delivered to the patient is about 10% to 30%.

In one embodiment, the step of providing a gas flow to the patient's airway may include providing the gas flow at a high flow rate. Moreover, the step of determining may include determining that the patient is spontaneously breathing with a patent airway if the waveform indicates at least one dip during a period of assumed respiratory cycle. Typically, each dip in the waveform represents a decrease in the fraction of oxygen with respect to a baseline fraction of oxygen delivered to the patient.

In some embodiments, the step of determining may include determining that the patient is spontaneously breathing with a patent airway if the waveform indicates at least one dip during a period of assumed respiratory cycle consistently over a plurality of respiratory cycles. In some embodiments, the step of determining may include determining that the patient is spontaneously breathing with a patent airway if the waveform consistently indicates at least one dip during a period of assumed respiratory cycle for a predetermined number of consecutive respiratory cycles, when the frequency of respiratory cycles is about 0.02 to 0.5 Hz (1 to 30 respiratory cycles per minute), or less than 0.5 Hz. The predetermined number of consecutive respiratory cycles may be at least 2 consecutive respiratory cycles, 5 or more consecutive respiratory cycles, or 2 to 5 consecutive respiratory cycles.

Advantageously, a determination of spontaneous breathing with a patent airway based on a consistent and repeating pattern in the waveform over a plurality of consecutive respiratory cycles may be more reliable, as it may discount the effect of noise or other erroneous signals. Providing the gas flow to the patient's airway may include providing the gas flow at any suitable flow rate. In one embodiment, the step of providing a gas flow to a patient's airway may include providing the gas flow at a high 30 flow rate of greater than 15 LPM. In particular, the step of providing a gas flow to a patient's airway includes providing the gas flow at a high flow rate of greater than 20 LPM.

In some embodiments, the step of providing a gas flow to a patient's airway may include providing the gas flow at a high flow rate of between about 20 LPM to 90 LPM. In some embodiments, the step of providing a gas flow to a patient's airway may include providing the gas flow at a high flow rate of between about 40 LPM to 70 LPM.

In this specification, “high flow” means, without limitation, any gas flow with a flow rate that is higher than usual/normal, such as higher than the normal inspiration flow rate of a healthy patient. Alternatively, or additionally, it can be higher than some other threshold flow rate that is relevant to the context—for example, where providing a gas flow to a patient at a flow rate to meet or exceed inspiratory demand, that flow rate might be deemed “high flow” as it is higher than a nominal flow rate that might have otherwise been provided. “High flow” is therefore context dependent, and what constitutes “high flow” depends on many factors such as the health state of the patient, type of procedure/therapy/support being provided, the nature of the patient (big, small, adult child) and the like. Those skilled in the art would know from context what constitutes “high flow”. It is a magnitude of flow rate that is over and above a flow rate that might otherwise be provided.

But, without limitation, some indicative values of high flow can be as follows.

In some configurations, delivery of gases to a patient at a flow rate of greater than or equal to about 5 or 10 litres per minute (5 or 10 LPM or L/min).

In some configurations, delivery of gases to a patient at a flow rate of about 5 or 10 LPM to about 150 LPM, or about 15 LPM to about 95 LPM, or about 20 LPM to about 90 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM. For example, a flow rate of gases supplied or provided to an interface via a system or from a flow source or flow modulator, may comprise, but is not limited to, flows of at least about 5, 10, 20, 30, 40, 50, 60, 70, 80, 90, 100, 110, 120, 130, 140, 150 LPM, or more, and useful ranges may be selected to be any of these values (for example, about 20 LPM to about 90 LPM, about 40 LPM to about 70 LPM, about 40 LPM to about 80 LPM, about 50 LPM to about 80 LPM, about 60 LPM to about 80 LPM, about 70 LPM to about 100 LPM, about 70 LPM to about 80 LPM).

In “high flow” the gas delivered will be chosen depending on for example the intended use of a therapy and/or respiratory support. Gases delivered may comprise a percentage of oxygen (Also referred to herein as fraction of oxygen). In some configurations, the percentage of oxygen in the gases delivered may be about 15% to about 100%, 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

Flow rates for “High flow” for premature/infants/paediatrics (with body mass in the range of about 1 to about 30 kg) can be different. The flow rate can be set to 0.4-8 L/min/kg with a minimum of about 0.5 L/min and a maximum of about 70 L/min. For patients under 2 kg maximum flow may be set to 8 L/min.

High flow has been found effective in meeting or exceeding the patient's normal real inspiratory flow, to increase oxygenation of the patient and/or reduce the work of breathing. Additionally, high flow therapy and/or respiratory support may generate a flushing effect in the nasopharynx such that the anatomical dead space of the upper airways is flushed by the high incoming gas flows. This creates a reservoir of fresh gas available of each and every breath, while minimising re-breathing of carbon dioxide, nitrogen, etc.

High flow may be used as a means to promote gas exchange and/or respiratory support through the delivery of oxygen and/or other gases, and through the removal of CO2 from the patient's airways. High flow may be particularly useful prior to, during or after a medical and/or anaesthetic procedure.

When used prior to a medical procedure, high gas flow can pre-load the patient with oxygen so that their blood oxygen saturation level and volume of oxygen in the lungs is higher to provide an oxygen buffer while the patient is in an apnoeic phase during the medical and/or anaesthetic procedure.

A continuous supply of oxygen can be useful for sustaining healthy respiratory function during medical procedures (such as during an anaesthetic procedure) where respiratory function might be compromised (e.g. diminishes or stops). When this supply is compromised, hypoxia and/or hypercapnia can occur. During anaesthetic procedures such as general anaesthesia where the patient is unconscious, the patient is monitored to detect when this happens. If oxygen supply and/or CO2 removal is compromised, the clinician may stop the medical procedure and facilitate oxygen supply and/or CO2 removal. This can be achieved for example by manually ventilating the patient through an anaesthetic bag and mask, or by providing a high flow of gases to the patient's airway using a high flow respiratory system.

Further advantages of high gas flow can include that the high gas flow increases pressure in the airways of the patient, thereby providing pressure support that opens airways, the trachea, lungs/alveolar and bronchioles. The opening of these structures enhances oxygenation, and to some extent assists in removal of CO2.

The increased pressure can also keep structures such as the larynx from blocking the view of the vocal chords during intubation. When humidified, the high gas flow can also prevent airways from drying out, mitigating mucociliary damage, and reducing risk of laryngospasms and risks associated with airway drying such as nose bleeding, aspiration (as a result of nose bleeding), and airway obstruction, swelling and bleeding. Another advantage of high gas flow is that the flow can clear smoke created during surgery in the air passages. For example, smoke can be created by lasers and/or cauterizing devices.

In some embodiments, the step of providing a gas flow to the patient's airway may include providing the gas flow at a low flow rate. Moreover, the step of determining may include determining that the patient is spontaneously breathing with a patent airway if the waveform indicates at least two dips during a period of assumed respiratory cycle. In some embodiments, the step of providing a gas flow to a patient's airway may include providing the gas flow at a low flow rate of less than 20 LPM. In particular, the step of providing a gas flow to a patient's airway may include providing the gas flow at a low flow rate of less than 15 LPM.

The method may further include, upon determining that the patient is apnoeic or has an obstructed airway based on the waveform, providing an indication that the patient is apnoeic or has an obstructed airway. Any suitable indication may be used. For example, the indication that the patient is apnoeic or has an obstructed airway includes any one or more of a sound indicator, a light indicator, and a display message. The sound indicator may include any suitable sound, for example a beep, a buzzer and/or an alarm or any combination thereof. The light indicator may include any suitable light, for example, an on/off light or flashing light. The display message may include text, for example a warning and/or alert message that the patient may be apnoeic or may have an obstructed airway.

The method may be performed during a medical procedure when the patient has a diminished respiratory function or a risk of diminished respiratory function resulting from anaesthetic agents. In one example, the medical procedure may be procedural sedation. In another example, the procedure may be general anaesthesia.

The method may be performed during patient monitoring, therapy, respiratory support or supplemental oxygen delivery. Additionally, or alternatively, the method may be performed in a hospital ward, in an intensive care unit (ICU) or emergency response vehicle.

The method may further include setting the gas flow at a continuous flow rate independent of the patient's breathing. In addition, the step of providing a gas flow to a patient's airway may include providing the gas flow at the continuous flow rate. Typically, a continuous flow rate refers a non-interrupted flow rate, or in other words a flow rate without any intermittent stopping and re-starting. The continuous flow rate may be any suitable flow rate. Moreover, the continuous flow rate may be a continuously varying flow rate, or a generally constant flow rate.

The step of providing the gas flow to the patient's airway may include continuously providing the gas flow to the patient's airway. Furthermore, the step of monitoring may include continuously monitoring the fraction of oxygen at the patient's airway.

The method may further include scaling a display of the waveform. In some embodiments, the method may include automatically scaling a display of the waveform when the waveform indicates that a change in oxygen fraction with respect to a baseline oxygen fraction delivered to the patient is less than about 30%. In particular, the method may further include automatically scaling a display of the waveform when the waveform indicates that a change in oxygen fraction with respect to a baseline oxygen fraction delivered to the patient is less than about 10%.

In some embodiments, the method may further include automatically scaling a display of the waveform when the waveform indicates that a baseline oxygen fraction delivered to the patient of less than a predetermined amount. For example, the method may further include automatically scaling a display of the waveform when the waveform indicates that a baseline oxygen fraction delivered to the patient of less than 1.

The scaling and automatic scaling of the display of the waveform may resize the waveform to advantageously allow a medical professional to more clearly distinguish between fluctuations in the waveform resulting from changes in the oxygen fraction and fluctuations in the waveform resulting from noise. In particular, the scaling of the display may allow the medical professional to change the scale, for example to enlarge (or zoom in to) a portion of the waveform, or zoom out to visualise the waveform over a greater number of assumed respiratory cycles.

Any scaling factor may be used during the step of automatic scaling. Typically, a scaling factor of 2 or more is used to resize the waveform. In one embodiment, the step of automatically scaling includes increasing the scale by a factor of 5 or more. The scaling factor may be used to increase or decrease the scale.

According to another aspect of the invention, there is provided a system of monitoring oxygen at a patient to determine breathing or patent airway, the system including
a flow source for providing a gas flow to a patient's airway, the gas flow including a predetermined fraction of oxygen,
one or more sensors for monitoring the fraction of oxygen at the patient's airway, and
one or more controllers being configured to generate a waveform representing the fraction of oxygen at the patient's airway based on input from the one or more sensors to allow for determination of whether the patient is spontaneously breathing with a patent airway.

The one or more controllers may be configured to determine whether the patient is spontaneously breathing with a patent airway. In particular, the one or more controllers may be configured to determine that the patient is spontaneously breathing with a patent airway when the waveform indicates a decrease in the fraction of oxygen with respect to a baseline fraction of oxygen delivered to the patient corresponding to an assumed respiration cycle of the patient. The one or more sensors may include an oxygen sensor.

The one or more controllers may be configured to determine that the patient is apnoeic or has an obstructed airway when the waveform indicates insufficient change in the fraction of oxygen over a period of time as measured at the patient's airway.

The system may further include a non-sealing patient interface for providing the gas flow to the patient's airway. Any suitable non-sealing patient interface may be provided. In one example, the non-sealing patient interface includes a non-sealing nasal cannula. In some embodiments, the system may provide a sealing patient interface.

The one or more sensors for monitoring the fraction of oxygen at the patient's airway may be adapted for monitoring the fraction of oxygen inside the patient's nasal passage, outside and proximate the patient's nasal passage, inside the patient's mouth, and/or outside and proximate the patient's mouth.

The system may further include a humidifier for humidifying the gas flow. The flow source may include a blower to facilitate movement of the gas flow. Moreover, the flow source may include an oxygen source.

The system may further include an inspiratory tube for providing the gas flow to the patient's airway. The inspiratory tube may include a heating element for providing heat to the gas flow.

In one embodiment, the one or more controllers may be configured to determine that the patient is spontaneously breathing with a patent airway if the waveform indicates a change in oxygen fraction with respect to a baseline oxygen fraction delivered to the patient of more than about 10%.

In one embodiment, the one or more controllers may be configured to determine that the patient is spontaneously breathing with a patent airway if the waveform indicates a change in oxygen fraction with respect to a baseline oxygen fraction delivered to the patient of more than about 30%.

In some embodiments, the one or more controllers may be further configured to determine that the patient is spontaneously breathing with a patent airway if the waveform indicates at least two dips during a period of assumed respiratory cycle when an inspiratory demand of the patient is not met. Typically, a dip in the waveform corresponds to a reduction in the fraction of oxygen at the patient's airway.

The one or more controllers may be further configured to determine that the patient is spontaneously breathing with a patent airway if the waveform indicates at least two dips during a period of assumed respiratory cycle when an inspiratory demand of the patient is not met, and the waveform indicates a change in oxygen fraction with respect to a baseline oxygen fraction delivered to the patient is about 10% to 30%.

In some embodiments, the flow source may provide the gas flow to the patient's airway at a high flow rate, and the one or more controllers may be configured to determine that the patient is spontaneously breathing with a patent airway if the waveform indicates at least one dip during a period of assumed respiratory cycle.

In some embodiments, the flow source may provide the gas flow to the patient's airway at a high flow rate of greater than 15 LPM. In some embodiments, the flow source may provide the gas flow to the patient's airway at a high flow rate of greater than 20 LPM. In some embodiments, the flow source may provide the gas flow to the patient's airway at a high flow rate of between about 20 LPM to 90 LPM. In some embodiments, the flow source may provide the gas flow to the patient's airway at a high flow rate of between about 40 LPM to 70 LPM.

In some embodiments, the flow source may provide the gas flow to the patient's airway at a low flow rate, and the one or more controllers may be configured to determine that the patient is spontaneously breathing with a patent airway if the waveform indicates at least two dips during a period of assumed respiratory cycle. In some embodiments, the flow source may provide the gas flow to the patient's airway at a low flow rate of less than 20 LPM. In some embodiments, the flow source may provide the gas flow to the patient's airway at a low flow rate of less than 15 LPM. In some embodiments, the flow source may provide the gas flow to the patient's airway at a low flow rate having a lower limit of about 0.5 LPM. In some embodiments, the flow source may provide the gas flow to the patient's airway at a low flow rate between about 2 to 6 LPM.

In some embodiments, one or more controllers may be configured to determine that the patient is spontaneously breathing with a patent airway if the waveform indicates at least one dip during a period of assumed respiratory cycle consistently over a plurality of respiratory cycles. In some embodiments, one or more controllers may be configured to determine that the patient is spontaneously breathing with a patent airway if the waveform consistently indicates at least one dip during a period of assumed respiratory cycle for a predetermined number of consecutive respiratory cycles, when the frequency of respiratory cycles is about 0.02 to 0.5 Hz (1 to 30 respiratory cycles per minute), or less than 0.5 Hz. The predetermined number of consecutive respiratory cycles may be at least 2 consecutive respiratory cycles, 5 or more consecutive respiratory cycles, or 2 to 5 consecutive respiratory cycles.

In particular, the one or more controllers may be configured to automatically determine that the patient is spontaneously breathing with a patent airway based on any one or more of the above-described parameters. Moreover, the one or more controllers may be configured to generate one or more signals to provide an indication in response to a determination that the patient is spontaneously breathing with a patent airway.

In some embodiments, the flow source may provide a gas flow at a continuous flow rate independent of the patient's breathing. In some embodiments, the flow source may continuously provide the gas flow to the patient's airway, and the one or more sensors may continuously monitor the fraction of oxygen at the patient's airway.

The one or more controllers may be further configured to provide an indication upon determining that the patient is apnoeic or has an obstructed airway based on the waveform. Any suitable system generated indication may be used. For example, the indication that the patient is apnoeic or has an obstructed airway may include any one or more of an alarm, a light indicator, and a display message.

In some embodiments, the system is a system of monitoring oxygen at a patient to determine spontaneous breathing with a patent airway during a medical procedure when the patient has a diminished respiratory function or a risk of diminished respiratory function resulting from anaesthetic agents. In one embodiment, the medical procedure includes procedural sedation. In one embodiment, the medical procedure includes general anaesthesia.

The system may further include an electronic display for displaying the waveform generated by the one or more controllers. The one or more controllers may be configured to automatically scale the display of the waveform when the waveform indicates that a change in oxygen fraction with respect to a baseline oxygen fraction delivered to the patient is less than about 30%. Optionally, the one or more controllers may be configured to automatically scale the display of the waveform when the waveform indicates that a change in oxygen fraction with respect to a baseline oxygen fraction delivered to the patient is less than about 10%.

The one or more controllers may be configured to automatically scale the display of the waveform by any suitable scaling factor. In some embodiments, the one or more controllers may be configured to automatically scale the display of the waveform by a factor between 2 to 10.

There is disclosed herein, a respiratory system comprising
gas delivery apparatus for delivering a gas flow to a patient, the gas flow including a predetermined fraction of oxygen,
one or more sensors for monitoring a fraction of oxygen at the patient, and
one or more controllers configured to
   receive input from the one or more sensors relating to the fraction of oxygen at the patient, and
   generate a display representing the fraction of oxygen at the patient,
wherein the one or more controllers is configured to determine when a change in the fraction of oxygen at the patient with respect to a baseline oxygen fraction delivered to the patient is less than a predetermined amount.

The one or more controllers may be configured to provide an indication to adjust the display upon determining that a change in the fraction of oxygen at the patient with respect to a baseline oxygen fraction delivered to the patient is less than the predetermined amount. In one embodiment, the one or more controllers may be configured to provide an indication to adjust the display upon determining that a baseline oxygen fraction delivered to the patient is less than a predetermined amount. For example, the one or more controllers may be configured to provide an indication to adjust the display upon determining that a baseline oxygen fraction delivered to the patient is less than 1.

Any suitable indication may be provided. For example, the one or more controllers may generate a signal to turn on a light indicator and/or sound indicator, or a display text message to suggest adjusting the display.

The one or more controllers may be configured to automatically adjust the display upon determining that a change in the fraction of oxygen at the patient with respect to a baseline oxygen fraction delivered to the patient is less than the predetermined amount.

In one embodiment, the baseline oxygen fraction is determined after monitoring one or more respiratory cycles. In another embodiment, the baseline oxygen fraction may be determined by applying an entrainment factor to the predetermined fraction of oxygen in the gas flow.

In some embodiments, the one or more controllers may be configured to automatically adjust the display when a change in the fraction of oxygen at the patient with respect to a baseline oxygen fraction delivered to the patient is less than 30%. In some embodiments, the one or more controllers may be configured to automatically adjust the display when a change in the fraction of oxygen at the patient with respect to a baseline oxygen fraction delivered to the patient is less than 10%.

The display generated by the one or more controllers may include a waveform of the monitored fraction of oxygen at the patient over time.

The one or more controllers may be configured to automatically adjust an axis of the waveform by a predetermined amount. For example, the one or more controllers may automatically adjust the vertical axis of the waveform and/or the horizontal axis of the waveform.

The one or more controllers may be configured to automatically adjust an axis of the waveform by any suitable amount. In one example, the one or more controllers may be configured to automatically adjust an axis of the waveform by a factor of 5 or more. Typically, the one or more controllers may be configured to automatically adjust an axis of the waveform by a factor inversely proportional to a change in the fraction of oxygen at the patient with respect to a baseline oxygen fraction delivered to the patient.

The gas delivery apparatus may deliver gas flow to the patient at flow rates of about 20 PLM to 90 PLM.

The one or more controllers may be configured to adjust the display when the flow rate meets or exceeds the patient's peak inspiratory demand.

In some embodiments, the gas delivery apparatus may include a non-sealing patient interface. In particular, the non-sealing patient interface may include a non-sealing nasal cannula.

In some embodiments, the respiratory system may further include a humidifier for humidifying the gas flow. The gas delivery apparatus may include a blower to facilitate movement of the gas flow. Moreover, the gas delivery apparatus may include an oxygen source. The respiratory system may further include an inspiratory tube for providing the gas flow to the patient's airway. The inspiratory tube may include a heating element for providing heat to the gas flow.

There is also disclosed herein, a method of determining spontaneous breathing with a patent airway, the method including the steps of obtaining sensor data representing a fraction of oxygen at a patient's airway when the patient is receiving a gas flow including a predetermined fraction of oxygen, generating a waveform representing the fraction of oxygen at the patient's airway, and analysing the waveform to determine an output indicative of whether the patient is spontaneously breathing with a patent airway.

There is also disclosed herein, a method of determining spontaneous breathing with a patent airway, the method including the steps of
obtaining sensor data representing a fraction of oxygen at a patient's airway when the patient is receiving a gas flow including a predetermined fraction of oxygen,
generating a waveform representing the fraction of oxygen at the patient's airway using the sensor data to enable determination of whether the patient is spontaneously breathing with a patent airway based on the waveform.

The method may further include determining whether the patient is spontaneously breathing with a patent airway.

The method may include generating a waveform using the sensor data such that changes in the fraction of oxygen for each respiratory cycle at the patient's airway as indicated by the waveform enables determination that the patient is spontaneously breathing with a patent airway.

The method may include generating a waveform using the sensor data such that a decrease in the fraction of oxygen with respect to a baseline fraction of oxygen delivered to the patient corresponding to an assumed respiration cycle of the patient as indicated by the waveform enables determination that the patient is spontaneously breathing with a patent airway.

The method may include generating a waveform using the sensor data such that an insufficient change in the fraction of oxygen over a period of time at the patient's airway as indicated by the waveform enables determination that the patient is apnoeic or has an obstructed airway.

The method may include automatically detecting that the patient is apnoeic or has an obstructed airway based on the waveform.

The method may include generating a waveform using the sensor data such that a change in oxygen fraction with respect to a baseline oxygen fraction delivered to the patient of more than about 10% as indicated by the waveform enables determination that the patient is spontaneously breathing with a patent airway.

The method may include generating a waveform using the sensor data such that a change in oxygen fraction with respect to a baseline oxygen fraction delivered to the patient of more than about 30% as indicated by the waveform enables determination that the patient is spontaneously breathing with a patent airway.

The method may include generating a waveform using the sensor data such that at least two dips during a period of assumed respiratory cycle when an inspiratory demand of the patient is not met as indicated by the waveform enables determination that the patient is spontaneously breathing with a patent airway.

The method may include generating a waveform using the sensor data such that at least two dips during a period of assumed respiratory cycle when an inspiratory demand of the patient is not met, and a change in oxygen fraction with respect to a baseline oxygen fraction delivered to the patient is about 10% to 30% as indicated by the waveform enables determination that the patient is spontaneously breathing with a patent airway.

The method may include generating a waveform using the sensor data such that at least one dip during a period of assumed respiratory cycle as indicated by the waveform when a gas flow at a high flow rate is provided to the patient's airway enables determination that the patient is spontaneously breathing with a patent airway. Each dip in the waveform may represent a decrease in the fraction of oxygen with respect to a baseline fraction of oxygen delivered to the patient.

The method may include generating a waveform using the sensor data such that at least two dips during a period of assumed respiratory cycle as indicated by the waveform when a gas flow at a low flow rate is provided to the patient's airway enables determination that the patient is spontaneously breathing with a patent airway.

The method may include generating a waveform using the sensor data such that at least one dip during a period of assumed respiratory cycle consistently over a plurality of respiratory cycles enables determination that the patient is spontaneously breathing with a patent airway.

The method may include generating a waveform using the sensor data such that at least one dip during a period of assumed respiratory cycle consistently over a predetermined number of consecutive respiratory cycles, when the frequency of respiratory cycles is about 0.02 to 0.5 Hz (1 to 30 respiratory cycles per minute), or less than 0.5 Hz enables determination that the patient is spontaneously breathing with a patent airway. The predetermined number of consecutive respiratory cycles may be at least 2 consecutive respiratory cycles, 5 or more consecutive respiratory cycles, or 2 to 5 consecutive respiratory cycles.

In order that the invention may be more readily understood and put into practice, one or more preferred embodiments thereof will now be described, by way of example only, with reference to the accompanying drawings.

### BRIEF DESCRIPTION OF DRAWINGS

FIG. 1 is a schematic diagram of a system of monitoring oxygen according to an embodiment of the invention.

FIGS. 2A and 2B are waveforms representing the fraction of oxygen as measured inside the patient's nose when the flow rate of gas flow is provided to the patient at a high flow rate and the patient's inspiratory demand is not met.

FIGS. 2C and 2D are also waveforms representing the fraction of oxygen as measured inside the patient's nose when the flow rate of gas flow is provided to the patient at a high flow rate and the patient's inspiratory demand is not met. The fraction of oxygen being delivered to the patient in FIGS. 2C and 2D is roughly half of that of FIGS. 2A and 2B.

FIGS. 3A and 3B are waveforms representing the fraction of oxygen as measured inside the patient's nose when the flow rate of gas flow is provided to the patient at a high flow rate and the patient's inspiratory demand is met.

FIGS. 3C and 3D are also waveforms representing the fraction of oxygen as measured inside the patient's nose when the flow rate of gas flow is provided to the patient at a high flow rate and the patient's inspiratory demand is met. The fraction of oxygen being delivered to the patient in FIGS. 3C and 3D is roughly half of that of FIGS. 3A and 3B.

FIGS. 4A and 4B are waveforms representing the fraction of oxygen as measured inside the patient's nose when the flow rate of gas flow is provided to the patient at a high flow rate and the patient's inspiratory demand is exceeded.

FIGS. 4C and 4D are also waveforms representing the fraction of oxygen as measured inside the patient's nose when the flow rate of gas flow is provided to the patient at a high flow rate and the patient's inspiratory demand is exceeded. The fraction of oxygen being delivered to the patient in FIGS. 4C and 4D is roughly half of that of FIGS. 4A and 4B.

FIGS. 5A and 5B are waveforms representing the fraction of oxygen as measured inside the patient's nose when the flow rate of gas flow is provided to the patient at a low flow rate and the patient's inspiratory demand is not met.

FIGS. 5C and 5D are also waveforms representing the fraction of oxygen as measured inside the patient's nose when the flow rate of gas flow is provided to the patient at a low flow rate and the patient's inspiratory demand is not met. The fraction of oxygen being delivered to the patient in FIGS. 5C and 5D is roughly half of that of FIGS. 5A and 5B.

FIGS. 6A to 6C are waveforms representing the fraction of oxygen measured outside and proximate the patient's mouth when the flow rate of gas flow is provided to the patient at a high flow rate and the patient's inspiratory demand is not met.

FIGS. 7A and 7B are waveforms representing the fraction of oxygen measured outside and proximate the patient's mouth when the flow rate of gas flow is provided to the patient at a high flow rate and the patient's inspiratory demand is met or exceeded.

FIG. 8 illustrates a waveform representing the fraction of oxygen measured outside and proximate the patient's mouth when the flow rate of gas flow is provided to the patient at a low flow rate and the patient's inspiratory demand is not met.

### DETAILED DESCRIPTION

There are a number of clinical situations where delivering a known fraction of inspired oxygen (that is “oxygenation”) is important (reference herein to “fraction” can be used interchangeably with the terms “concentration” and “proportion”). For example, during an oxygenation phase (commonly referred to as pre-oxygenation) before general anaesthesia, it is desirable to administer a fraction of inspired oxygen to ensure that the lungs contain as much oxygen as possible. A further example is that it is desirable to administer a known fraction of inspired oxygen to patients being treated for respiratory distress. In another more general example, respiratory support may be provided to a patient—e.g. respiratory support provided to a patient using a gas flow with a high flow rate (“high flow”).

Where it is desirable to deliver a flow of gas to oxygenate (that is to deliver oxygen to meet the oxygen needs of) a patient, the flow of gas can be provided with desired oxygen concentration (that is usually above the oxygen concentration of ambient air). For example, this might be in situations where ambient air cannot meet the oxygenation needs of the patient, as ambient air may not have a high enough oxygen concentration to effectively oxygenate the patient. This can be achieved by operating a respiratory system to deliver a flow of gas with an oxygen concentration that is higher than the oxygen concentration found in ambient air such that the flow of gas meets the oxygenation needs of the patient. The flow rate of the flow of gases may additionally or alternatively be increased.

Monitoring gases at the patient's airway when the patient is receiving respiratory support can provide crucial feedback to clinicians. According to embodiments of the present invention, a respiratory system can be used to monitor the fraction of oxygen at the patient's airway to allow prompt determination of whether the patient is spontaneously breathing with a patent airway. Patient monitoring of this kind can be particularly useful when the patient has diminished respiratory function, for example during a medical procedure (e.g. procedural sedation) when anaesthetic agents are used.

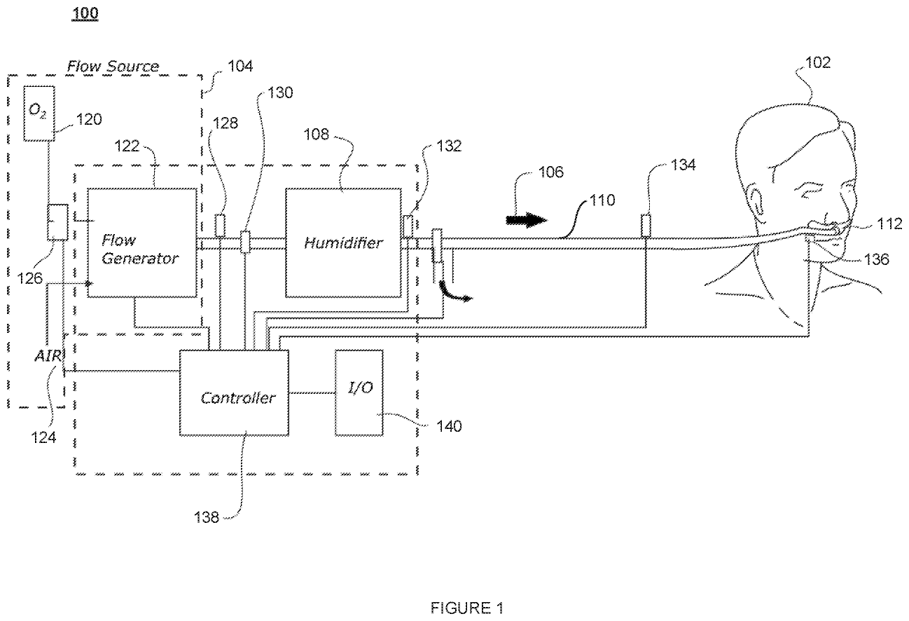

FIG. 1 shows a respiratory system 100 that can provide a flow of gas (with a known oxygen fraction) for providing respiratory support (preferably oxygenation) to a patient 102 at any suitable flow rate. In some configurations, the flow of gas has an oxygen concentration required by the patient and a flow rate that meets the inspiratory demand of the patient. There is usually considerable inter-patient and intra-patient variability in inspiratory demand caused by a variety of factors including anatomy, physiology, anxiety, level of consciousness, type or amount of any anaesthetic agent given, and respiratory disease state.

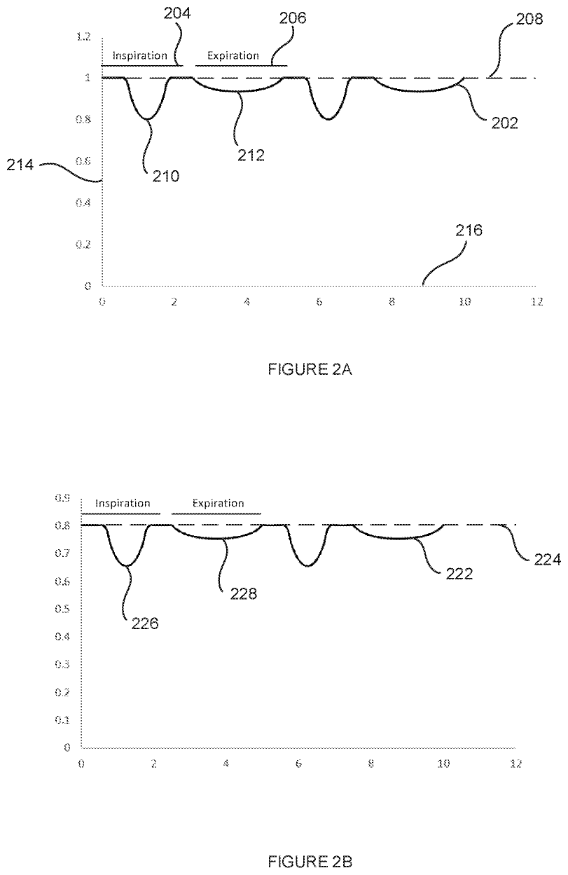

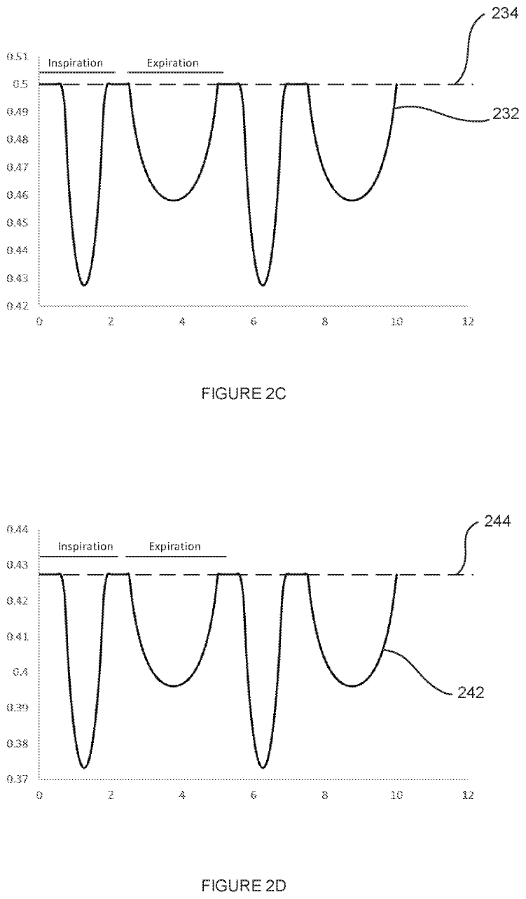

If the flow rate of gas being delivered to a patient does not meet inspiratory demand, then entrainment of ambient air can occur. Entrainment of ambient air may occur via the patient's nose and/or mouth. When entrainment occurs, the concentration of the constituent gas will be altered due to the different concentration of that constituent gas in ambient air, thereby affecting a baseline fraction of oxygen measured at the patient's airway. As described in further detail below with reference to FIGS. 2A to 5D, different waveforms can be generated by the system 100 in different scenarios covering different flow rates of gas flow with respect to the patient's inspiratory demand. The waveforms represent the fraction of oxygen at the patient's airway in the different scenarios, to allow determination of whether the patient 102 is spontaneously breathing with a patent airway.

Now returning to FIG. 1, the respiratory system 100 comprises a flow source 104 for providing a gas flow 106 at a predetermined flow rate. The gas flow 106 may include pure oxygen (e.g. having a fraction of oxygen of 1, or concentration of oxygen of 100%), or a mixture of oxygen and one or more other gases. In alternative embodiments, the respiratory system 100 may have a connection for coupling to a remote flow source (not shown). As such, the flow source 104 may form part of the respiratory system 100 or may be provided separately to the respiratory system 100. In some embodiments, the flow source may include a plurality of separate components, some flow source components forming part of the respiratory system 100 and some components being provided separately to the system 100.

In the embodiment shown in FIG. 1, the respiratory system 100 may include gas delivery apparatus for delivering a gas flow to the patient 102. In particular, the system 100 may include a flow source 104, a humidifier 108 for humidifying the gas flow 106, an inspiratory tube 110, conduit (e.g. dry line or heated breathing tube), patient interface 112, pressure relief valve, and filter.

The flow source 104 could be an in-wall supply of oxygen, a tank of oxygen 120, a tank of other gas and/or a flow apparatus with a flow generator 122. FIG. 1 shows a flow source 104 including a flow generator 122, an optional air inlet 124, and optional connection to an oxygen source (such as tank or O2 generator) 120 via a shut off valve and/or regulator and/or other gas flow controller 126, but this is just one option. The flow generator 122 can control flows delivered to the patient 102 using one or more valves, or optionally the flow generator 122 can comprise a blower (not shown) to facilitate movement of the gas flow 106. The flow source 104 could be one or a combination of a flow generator 122, oxygen source 120, air source 124 as described. The flow source 122 is shown as part of the respiratory system 100, although in the case of an external oxygen tank or in-wall source, it may be considered a separate component, in which case the respiratory system 100 has a connection port to connect to such a flow source. The flow source provides a flow of gas that can be delivered to a patient via a delivery conduit 110, and patient interface 112.

The patient interface 112 may be an unsealed (non-sealing) interface (for example when used in high flow therapy) such as a non-sealing nasal cannula, or a sealed (sealing) interface (for example when used in CPAP) such as a nasal mask, full face mask, or nasal pillows. In some embodiments, the patient interface 112 is a non-sealing patient interface which would for example help to prevent barotrauma (e.g. tissue damage to the lungs or other organs of the respiratory system due to difference in pressure relative to the atmosphere). In some embodiments, the patient interface 112 is a sealing mask that seals with the patient's nose and/or mouth. The patient interface 112 may be a nasal cannula with a manifold and nasal prongs, and/or a face mask, and/or a nasal pillows mask, and/or a nasal mask, and/or a tracheostomy interface, or any other suitable type of patient interface. The flow source 104 could provide a base gas flow rate of between, e.g. 0.5 litres per minute (LPM) and 375 litres per minute (LPM), or any range within that range, or even ranges with higher or lower limits, as previously described.

The flow source 104 can provide the gas flow 106 at any suitable flow rate depending on requirements of the patient and/or relevant medical treatment(s). In some embodiments, the flow source 104 may provide the gas flow 106 at a high flow rate of greater than 15 LPM, or greater than 20 LPM, or between about 20 LPM to 90 LPM, or between about 40 LPM to 70 LPM. In some embodiments, the flow source 104 may provide the gas flow 106 at a low flow rate of less than 20 LPM, or less than 15 LPM.

Typically, the flow rate of the gas flow 106 can be provided at a continuous flow rate. In particular, the flow rate of the gas flow 106 can be provided at a continuous flow rate independent of the patient's breathing. The continuous flow rate can be variable or generally constant.

A humidifier 108 can optionally be provided between the flow source 104 and the patient 102 to provide humidification of the delivered gas 106. In some embodiments, a humidifier may be provided as part of the flow source 104. In particular, the flow generator 122 may include a built-in humidifier. Humidification in the gas flow 106 can allow the comfortable delivery of gas flow at low and/or high flow rates. Humidity in the delivered gas flow also prevents the patient's airway from drying out, thereby mitigating mucociliary damage, and reducing risk of laryngospasms. Humidity in the gas flow can also reduce risks associated with airway drying such as nose bleeding, aspiration (as a result of nose bleeding), and airway obstruction, swelling and bleeding. It can also prevent a laryngoscope from getting stuck to the skin of the patient in a dry airway, which can cause trauma to the patient. In some configurations the gas flow may be humidified to contain greater than 10 mg/L of water, or greater than 20 mg/L, or greater than 30 mg/L, or up to 44 mg/L. In some configurations the gas flow may be heated by a heater (not shown) to 21° C. to 42° C., or 25° C. to 40° C., or 31° C. to 37° C., or about 31° C., or about 37° C.

One or more sensors 128, 130, 132, 134 such as flow, oxygen fraction, pressure, humidity, temperature or any other suitable sensors can be placed throughout the system 100 and/or at, on or near the patient 102. Alternatively, or additionally, sensors from which such parameters can be derived could be used. In addition, or alternatively, the sensors 128 to 134 can be one or more physiological sensors for sensing patient physiological parameters such as, blood pressure, heart rate, oxygen saturation, partial pressure of oxygen in the blood, respiratory rate, end tidal carbon dioxide, partial pressure of carbon dioxide in the blood. Alternatively, or additionally, sensors from which such parameters can be derived could be used. Other patient sensors could comprise EEG sensors, torso bands to detect breathing, and any other suitable sensors. In some configurations the humidifier may be optional, or it may be preferred due to the advantages of humidified gases helping to maintain the condition of the airways. One or more of the sensors might form part of the respiratory system 100, or be external thereto, with the respiratory system 100 having inputs from any external sensors. The sensors can be configured for communication with a controller 138.

In the specific embodiment illustrated in FIG. 1, a sensor 136 is provided for measuring the oxygen fraction at the patient 102. This can be placed on the patient interface 112, for example, to measure or otherwise allow determination of the fraction of oxygen at the patient's airway (e.g. inside or outside/proximate the mouth and/or nose). The sensor 136 may be measuring/sampling the fraction of oxygen proximate the patient's airway continuously/periodically so as to continuously monitor the fraction of oxygen at the patient's airway. Typically, the system 100 continuously monitors the fraction of oxygen at the patient's airway whilst the system 100 provides a gas flow to the patient 102.

The system 100 further includes a controller 138 configured for communication with the sensor 136 and to receive input from the sensor 136 to allow determination of the fraction of oxygen at the patient 102. Based on information from the sensor 136, the controller 138 is able to monitor the fraction of oxygen at the patient's airway, and generate a waveform representing the fraction of oxygen at the patient's airway for display, for example on a display device (not shown) associated with an input/output interface (user interface) of controller 138. Some typical waveforms generated by the controller 138 based on input from the sensor 136 for display on a display device are illustrated in FIGS. 2A to 5D.

As explained in further detail below, the waveforms facilitate determination of whether the patient is spontaneously breathing with a patent airway. In particular, the waveform typically indicates changes in the fraction of oxygen for each respiratory cycle as measured at the patient's airway when the patient is spontaneously breathing with a patent airway. In the event that the waveform indicates insufficient changes in the fraction of oxygen, it may be determined either manually by a clinician or automatically by the controller 138, that the patient may be apnoeic or may have an obstructed airway. In the event that the controller 138 makes the determination that the patient may be apnoeic or may have an obstructed airway, the controller 138 may direct the user interface 140 to provide a suitable indication.

The user interface 140 may include one or more indicators for providing an indication that the patient may be apnoeic or may have an obstructed airway. The one or more indicators may include any one or more of a sound indicator (e.g. buzzer, alarm), a light indicator (e.g. an LED light), or both. In addition, or alternatively, the controller 138 may generate a text message for display on the display device of the user interface 140.

Moreover, the user interface 140 may receive information from a user (e.g. clinician or patient) that can be used for determining oxygenation requirements, anaesthetic gas agent and/or flow rates of the gas flow 106. For example, but not limiting, the user interface 140 can be used to receive manual input relating to the fraction of oxygen at the patient's airway and/or flow rate of the flow of gases to be provided to the patient. The respiratory system 100 can also be operatively configured to determine dose/oxygenation requirements (hereinafter “oxygen requirements”) of a patient for/in relation to anaesthesia (that is, the oxygen requirements pre-anaesthesia during a pre-oxygenation phase and/or the oxygen requirements during anaesthesia-which might include when the patient is apnoeic or when the patient is breathing), as well as after such a procedure.

In some embodiments, the respiratory system 100 can be configured to provide high flow gas to a patient and adjust the parameters of the high flow gas (such as pressure, flow rate, volume of gas, gas composition) delivered to the patient as required to meet oxygenation requirements, for example based on a determined oxygen fraction at the patient's nose and/or mouth.

The controller 138 can be configured to operate the respiratory system 100 so that the gas flow 106 has a flow rate with an oxygen fraction that meets the patient's requirements and provides the required therapy. The oxygen fraction may be a known oxygen fraction. For example, where pre-oxygenation of a patient prior to administration of anaesthesia is desired, the controller 138 can operate the respiratory system 100 to provide a gas flow 106 with an oxygen fraction of at or about 100%. In another example, where sedation of a patient is desirable, the controller 138 can operate the respiratory system 100 to provide a gas flow 106 with an oxygen fraction of about 21% or more during the sedation procedure. Preferably, the oxygen fraction of the gas flow provided during a sedation procedure is more than 21%, for example about 30% or about 50% or more. If the patient becomes apnoeic during the sedation procedure, the controller 138 or clinician can adjust the oxygen fraction of the gas flow to anywhere between about 21% and about 100%. Preferably, the controller 138 will increase the oxygen fraction in the gas flow 106, preferably to an oxygen fraction greater than a previous oxygen fraction, but this could be done manually. The fraction of oxygen in the gas flow 106 can be controlled in any suitable way, for example by controlling a valve coupled to the oxygen source 120 to increase/decrease the amount of oxygen relative to ambient gas flow to control the proportion (concentration) of oxygen in the total gas flow 106.

Whilst FIG. 1 illustrates a single controller 138, it is understood that the respiratory system may include one or more controllers, and/or be configured to interface with one or more controllers external to the respiratory system (e.g. through a network connection). Indeed, the controller 138 can also include one or more processors to control operations of the respiratory system 100.

The respiratory system 100 could be an integrated or a separate component-based arrangement, generally shown in the dotted box in FIG. 1. In some configurations, the respiratory system could be a modular arrangement of components. Furthermore, the respiratory system may comprise some of the components shown, not necessarily all are essential. Also, the conduit and patient interface do not have to be part of the system 100 and could be considered separate. Hereinafter it will be referred to as respiratory system 100, but this should not be considered limiting. Respiratory system 100 will be broadly considered herein to comprise anything that provides a flow rate of gas to a patient 102 with which a monitoring system can be used to monitor the fraction of oxygen at the patient's airway and generate a waveform representing the fraction of oxygen at the patient's airway.

The respiratory system 100 may be used for any suitable oxygenation purpose including, without limitation, pre-oxygenation during an anaesthetic procedure (e.g. anaesthesia or sedation), after anaesthetic or sedative agents are administered to a patient during an anaesthetic procedure (e.g. anaesthesia or sedation; as per the disclosure of PCT applications WO2016/157102, and WO2016/133406 (US equivalents being US20180280641 and US20180126110 respectively) which are incorporated herein in their entirety) for example, high flow respiratory support, high flow therapy, ventilation, provision of high flow gas-flows or anywhere else monitoring of the patient to determine spontaneous breathing with a patent airway is desired, for example in the hospital ward, ICU or emergency response vehicle. The respiratory system 100 may be used during patient monitoring, therapy, respiratory support or supplemental oxygen delivery. Now turning to FIG. 2A, which illustrates a waveform 202 representing the fraction of oxygen as measured inside the patient's nasal passage over time. In particular, the waveform 202 represents the fraction of oxygen as measured inside the patient's nose over two respiratory cycles. For waveform 202, the patient's mouth may be substantially closed. Each respiratory cycle including an inspiration phase 204 and expiration phase 206. The gas flow 106 provided by the system 100 in FIG. 2A is 100% oxygen (fraction of oxygen, FiO₂=1). In the scenario illustrated in FIG. 2A, the baseline fraction of oxygen 208 delivered to the patient 102 is also 1 (FiO_{2(baseline)}=1). In some cases, the baseline fraction of oxygen may be less than the fraction of oxygen provided by the system 100, for example due to entrainment of gases at the patient interface, particularly when a non-sealing patient interface is used (as shown in FIG. 2B). The baseline fraction of oxygen may also be less than the fraction of oxygen provided by the system 100 if the gas flow delivered to the patient is humidified.

FIG. 2A illustrates a generated waveform 202 in a scenario where the flow rate of the gas flow 106 is provided at a high flow rate, for example 30 LPM and the patient's peak inspiratory demand is determined to be roughly 40 LPM. In this scenario, the flow rate of the gas flow 106 does not meet the peak inspiratory demand of the patient 102.

The waveform 202 indicates a decrease in the fraction of oxygen (as presented by a dip 210, 212 in the waveform) corresponding to each inspiration 204 and expiration 206 phase of a respiratory cycle. When the patient's inspiratory demand is not being met, the waveform 202 indicates two dips 210, 212 during a period of assumed respiratory cycle. Each dip 210, 212 in the waveform 202 represents a decrease in the fraction of oxygen at the patient 102 with respect to the baseline 208. Dip 210 in the waveform 202 is caused by entrainment during inspiration as the patient's inspiratory demand is not met. Dip 212 in the waveform 202 is caused by the patient's exhalation mixing with the delivered gas flow.

In particular, the first dip 210 of the waveform 202 corresponding to the inspiration phase of the respiratory cycle indicates a decrease in the fraction of oxygen to about 0.8 (FiO₂₍ᵢₙₛₚᵢᵣₐₜᵢₒₙ₎=0.8), which is a reduction from the baseline 208 of 20%. The second dip 212 of the waveform 202 corresponding to the expiration phase of the respiratory cycle indicates a decrease in the fraction of oxygen to about 0.9 (FiO₂₍ₑₓₚᵢᵣₐₜᵢₒₙ₎=0.9), which is a reduction from the baseline 208 of roughly 10%. For the waveform 202, the overall change in oxygen fraction with respect to the baseline 208 is about 20%, as represented by the larger dip 210 corresponding to the inspiration phase of the respiratory cycle.

Typically, when the patient's inspiratory demand is not being met, it can be determined that the patient is spontaneously breathing with a patent airway if the waveform indicates a change in oxygen fraction with respect to a baseline oxygen fraction delivered to the patient of more than about 10%, or more than 30%, or between 10% to 30%.

Conversely, a waveform indicating insufficient change in the oxygen fraction with respect to the baseline oxygen fraction delivered to the patient may indicate that the patient is apnoeic or has an obstructed airway.

For example, a waveform indicating a change in oxygen fraction of 0% or near 0% with respect to the baseline oxygen fraction delivered to the patient may indicate that the patient is apnoeic or has an obstructed airway. It is noted that the waveform may indicate a change in oxygen fraction of 0% or near 0% with respect to the baseline oxygen fraction delivered to the patient in some situations when the patient is spontaneously breathing, for example when the patient's peak inspiratory demand is met and the fraction of oxygen is measured inside or proximate the patient's nasal passage, and the patient's expiratory flow is predominantly exiting via the patient's mouth. In such a scenario, the clinician may move the sensor 136 to measure the fraction of oxygen inside or outside/proximate the patient's mouth.

In another example, a waveform indicating notable decreases in the percentage change in the fraction of oxygen at the patient's airway over time (i.e. as monitored over a number of respiratory cycles) may indicate that the patient has a partially obstructed airway.

In some embodiments, user interface 140 may allow adjustment of the display to change the scale 214, 216 of the waveform 202. For example, the user interface 141 may allow a clinician to change the scale 214, 216 to effectively ‘zoom in’ on the waveform to more clearly visualise the waveform and distinguish between changes in the fraction of oxygen due to the patient and changes in the fraction of oxygen due to noise. Similarly, the user interface 141 may allow a clinician to change the scale 214, 216 to effectively ‘zoom out’ on the waveform to more clearly visualise changes in the waveform over a greater number of respiratory cycles, for example to facilitate the identification of patterns in the waveform over time. For instance, allowing a clinician to ‘zoom out’ on the waveform may facilitate the determination of whether there are notable decreases in the percentage change in the fraction of oxygen at the patient's airway over time to determine whether the patient has a partially obstructed airway. The adjustment may include adjustment of the vertical axis 214, which represents the fraction of oxygen, and/or adjustment of the horizontal axis 216, which represents elapsed time or number of assumed respiratory cycles.

In some embodiments, the controller 138 may determine when a change in the fraction of oxygen at the patient 102 with respect to a baseline oxygen fraction 208 delivered to the patient is less than a predetermined amount, and/or that a baseline oxygen fraction 208 delivered to the patient is less than a predetermined amount. Once the controller 138 has made the determination(s), the controller 138 can provide an indication to adjust the display. The indication can be in any suitable form, for example a light indication (e.g. a light may turn on or flash), a sound indication (a buzzer may generate a beep), or a text message may be generated for display suggesting adjustment of the scale 214, 216.

In some embodiments, the controller 138 may automatically adjust the display upon determining that a change in the fraction of oxygen at the patient with respect to a baseline oxygen fraction delivered to the patient is less than the predetermined amount. The threshold for the change in the fraction of oxygen with respect to the baseline which prompts the automatic scale adjustment can be set to any suitable range. For example, the controller 138 may automatically scale the display of the waveform when a change in oxygen fraction with respect to a baseline oxygen fraction delivered to the patient is less than about 30%, or less than about 10%. In some embodiments, the controller 138 may automatically adjust the display upon determining that a baseline fraction of oxygen delivered to the patient is less than 1.

The automatic scaling can be set to any suitable amount, for example, the controller 138 may automatically increase the scale by a factor of 2 or more, or a factor of 5 or more. In some embodiments, the controller 138 may automatically adjust the display based on a predetermined correlation. For example, the controller 138 may be configured to automatically adjust one or both axes of the waveform by a factor inversely proportional to a change in the fraction of oxygen at the patient with respect to a baseline oxygen fraction delivered to the patient.

Now referring to FIG. 2B, which illustrates a waveform 222 representing the fraction of oxygen as measured inside the patient's 102 nose over time in a scenario similar to that of FIG. 2A. For waveform 222, the patient's mouth may be substantially closed. The flow rate of the gas flow 106 is provided at a high flow rate, for example 30 LPM and the patient's peak inspiratory demand is determined to be roughly 40 LPM. Accordingly, the flow rate of the gas flow 106 does not meet the peak inspiratory demand of the patient 102.

The baseline fraction of oxygen 224 delivered to the patient 102 is roughly 0.8 (FiO_{2(baseline)}=0.8). The baseline fraction of oxygen may be less than the fraction of oxygen provided by the system 100, for example due to entrainment of gases at the patient interface. Similarly to FIG. 2A, the waveform 222 indicates a decrease in the fraction of oxygen (as presented by dips 226, 228 in the waveform) corresponding to each assumed respiratory cycle. When the patient's inspiratory demand is not being met, the waveform 222 indicates two dips 226, 228 during a period of assumed respiratory cycle. Each dip 226, 228 in the waveform 222 represents a decrease in the fraction of oxygen at the patient 102 with respect to the baseline 224. Dip 226 in the waveform 222 is caused by entrainment during inspiration as the patient's inspiratory demand is not met. Dip 228 in the waveform 222 is caused by the patient's exhalation mixing with the delivered gas flow.

Typically, the baseline oxygen fraction 224 (FiO_{2(baseline)}) can be determined after monitoring one or more respiratory cycles. In some embodiments, the baseline oxygen fraction (FiO_{2(baseline)}) is determined by applying an entrainment factor to the predetermined fraction of oxygen in the gas flow. In some embodiments, the baseline oxygen fraction (FiO_{2(baseline)}) is set by a user.

Now referring to FIGS. 2C and 2D, which illustrates waveforms 232, 242 representing the fraction of oxygen as measured inside the patient's 102 nose over time in a scenario similar to that of FIGS. 2A and 2B. The patient's mouth may be substantially closed. The flow rate of the gas flow 106 is provided at a high flow rate, for example 30 LPM and the patient's peak inspiratory demand is determined to be roughly 40 LPM. Accordingly, the flow rate of the gas flow 106 does not meet the peak inspiratory demand of the patient 102.

For FIG. 2C, the baseline fraction of oxygen 234 delivered to the patient 102 is roughly 0.5 (FiO_{2(baseline)}=0.5). For FIG. 2D, the baseline fraction of oxygen 244 delivered to the patient 102 is roughly 0.428 (FiO_{2(baseline)}=0.428). The waveforms 232, 242 each indicate a decrease in the fraction of oxygen (as presented by dips in the waveform) corresponding to each assumed respiratory cycle.

It has been observed that in the scenario illustrated in FIGS. 2A and 2B, each of the waveforms 202, 222 indicates a change in oxygen fraction with respect to their respective baseline oxygen fraction 208, 224 delivered to the patient of about 20% and 18% respectively. In the scenarios illustrated in FIGS. 2C and 2D, each of the waveforms 232, 242 indicates a change in oxygen fraction with respect to their respective baseline oxygen fraction 234, 244 delivered to the patient of about 14% and 13% respectively. Based on the waveforms 202, 222, 232, 242 illustrated in FIGS. 2A to 2D, it can be determined that the patient 102 is spontaneously breathing with a patent airway.

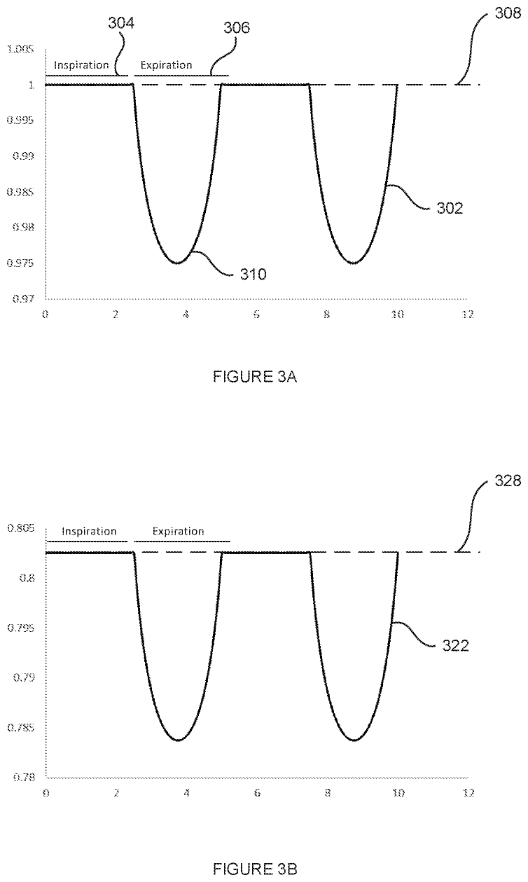

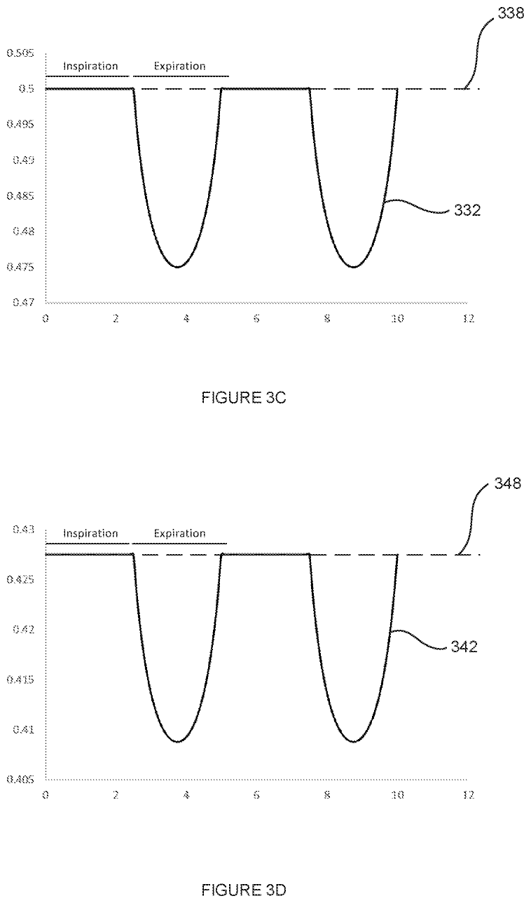

FIGS. 3A to 3D each illustrate waveforms 302, 322, 332, 342 representing a fraction of oxygen as measured inside the patient's 102 nose over two assumed respiratory cycles. The patient's mouth may be substantially closed. As more clearly shown in FIG. 3A, each respiratory cycle including an inspiration phase 304 and expiration phase 306. The waveforms 302, 322, 332, 342 are generated in a scenario where the flow rate of the gas flow 106 is provided at a high flow rate, for example 30 LPM and the patient's peak inspiratory demand is determined to be roughly 30 LPM. In this scenario, the flow rate of the gas flow 106 meets the peak inspiratory demand of the patient 102.

The gas flow 106 provided by the system 100 in FIG. 3A is 100% oxygen (fraction of oxygen, FiO₂=1). Similar to FIG. 2A, this is a scenario in which the baseline fraction of oxygen 308 delivered to the patient 102 is also 1 (FiO_{2(baseline)}=1). In some cases, the baseline fraction of oxygen is less than the fraction of oxygen provided by the system 100. For example, in FIGS. 3B to 3D, baseline fraction of oxygen 328, 338, 348 delivered to the patient 102 is roughly 0.803, 0.5, 0.428 respectively (FiO_{2(baseline)}=0.803, 0.5, 0.428).

Each of the waveforms 302, 322, 332, 342 indicate changes in the fraction of oxygen as presented by dips in the respective waveforms. For example, as illustrated in FIG. 3A, the waveform 302 indicates one dip 310 corresponding to an assumed expiration 306 phase of a respiratory cycle. Typically, when the patient's inspiratory demand (optionally peak inspiratory demand) is met, the waveform 302 will not indicate a reduction in the fraction of oxygen during an inspiration phase of each respiratory cycle, as entrainment of ambient air generally would not occur. When the patient's inspiratory demand is being met, the waveform 302 indicates one dip 310 during a period of assumed respiratory cycle. Each dip 310 in the waveform 302 represents a decrease in the fraction of oxygen at the patient 102 with respect to the baseline 308. Dip 310 in the waveform 302 is caused by the patient's exhalation mixing with the delivered gas flow.

In some instances, it has been observed that when the inspiratory demand is met or exceeded, the waveform may indicate a dip (not shown) corresponding to an inspiratory phase of the assumed respiratory cycle. Such observations may be made when the patient is receiving a gas flow via the nose at a high flow rate that meets or exceeds the patient's inspiratory demand, and the sensor 136 is positioned inside, or outside and proximate the patient's mouth, and the patient is partially breathing through the mouth. In this scenario, the sensor 136 may detect a reduction in the fraction of oxygen during an inspiratory phase of the patient's respiratory cycle, corresponding to a dip in the waveform during an inspiratory phase of an assumed respiratory cycle. Accordingly, in some embodiments, when the patient's inspiratory demand is met or exceeded, the waveform may indicate two dips during a period of assumed respiratory cycle.

It has been observed that in the scenarios illustrated in FIGS. 3A and 3B, each of the waveforms 302, 322 indicates a change in oxygen fraction with respect to their respective baseline oxygen fraction 308, 328 delivered to the patient of about 2.5% and 2.3% respectively. In the scenarios illustrated in FIGS. 3C and 3D, each of the waveforms 332, 342 indicates a change in oxygen fraction with respect to their respective baseline oxygen fraction 338, 348 delivered to the patient of about 5% and 4% respectively. Based on the waveforms 302, 322, 332, 342 illustrated in FIGS. 3A to 3D, it can be determined that the patient 102 is spontaneously breathing with a patent airway.

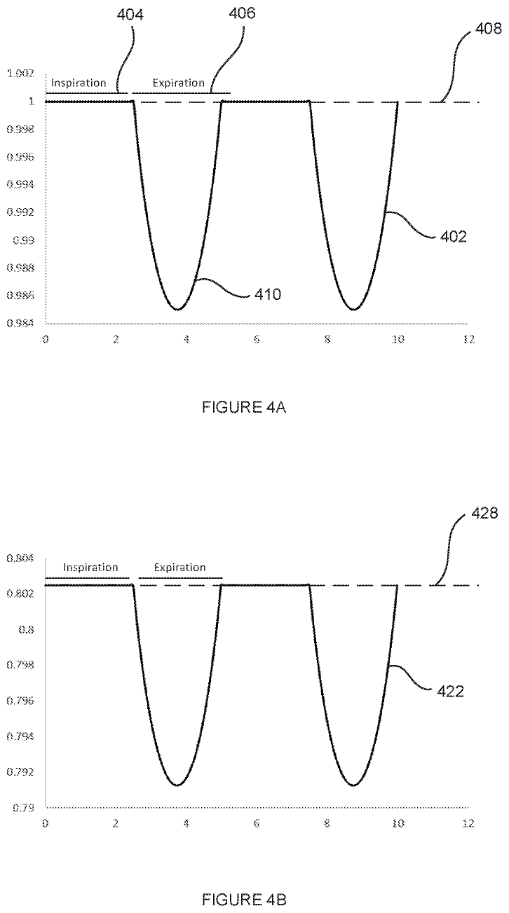

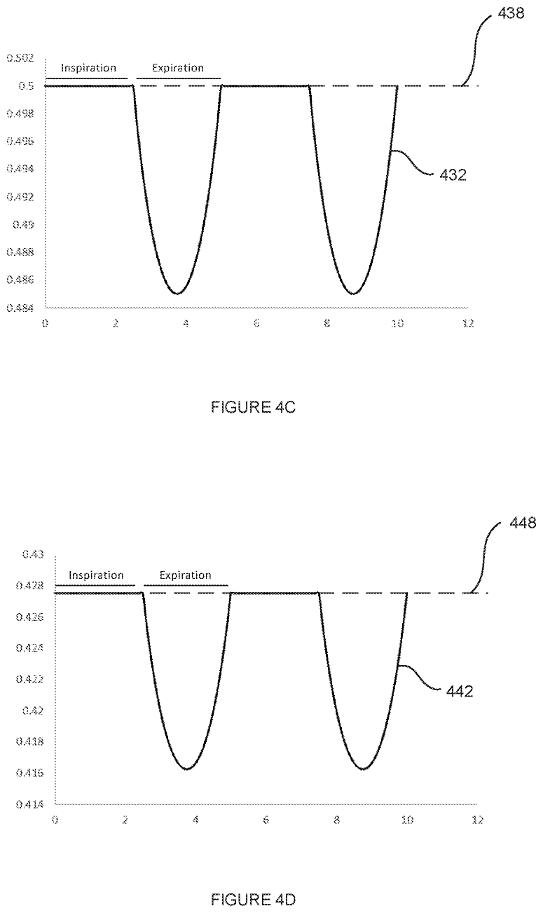

FIGS. 4A to 4D each illustrate waveforms 402, 422, 432, 442 representing a fraction of oxygen as measured inside the patient's 102 nose over two assumed respiratory cycles. The patient's mouth may be substantially closed. As more clearly shown in FIG. 4A, each respiratory cycle includes an inspiration phase 404 and expiration phase 406. The waveforms 402, 422, 432, 442 are generated in a scenario where the flow rate of the gas flow 106 is provided at a high flow rate, for example 70 LPM and the patient's peak inspiratory demand is determined to be roughly 30 LPM. In this scenario, the flow rate of the gas flow 106 exceeds the peak inspiratory demand of the patient 102.

The gas flow 106 provided by the system 100 in FIG. 4A is 100% oxygen (fraction of oxygen, FiO₂=1). Similar to FIGS. 2A and 3A, this is a scenario in which the baseline fraction of oxygen 408 delivered to the patient 102 is also 1 (FiO_{2(baseline)}=1). In some cases, the baseline fraction of oxygen may be less than the fraction of oxygen provided by the system 100. For example, in FIGS. 4B to 4D, baseline fraction of oxygen 428, 438, 448 delivered to the patient 102 is roughly 0.802, 0.5, 0.428 respectively (FiO_{2(baseline)}=0.802, 0.5, 0.428).

Each of the waveforms 402, 422, 432, 442 indicate changes in the fraction of oxygen as presented by dips in the respective waveforms. For example, as illustrated in FIG. 4A, the waveform 402 indicates one dip 410 corresponding to an assumed expiration phase 406 of a respiratory cycle. Typically, when the patient's inspiratory demand is met or exceeded, the waveform 402 will not indicate a reduction in the fraction of oxygen during an inspiration phase of each respiratory cycle, as entrainment of ambient air would typically not occur. When the inspiratory demand is being exceeded, the waveform 402 indicates one dip 410 during a period of assumed respiratory cycle. Each dip 410 in the waveform 402 represents a decrease in the fraction of oxygen at the patient 102 with respect to the baseline 408. Dip 410 in the waveform 402 is caused by the patient's exhaled breath mixing with the delivered gas flow. As previously mentioned, in some scenarios, a dip (not shown) may be observed corresponding to an inspiration phase of the respiratory cycle. In these cases, when the inspiratory demand is being exceeded, the waveform may indicate two dips during a period of assumed respiratory cycle,

It has been observed that in the scenarios illustrated in FIGS. 4A and 4B, each of the waveforms 402, 422 indicates a change in oxygen fraction with respect to their respective baseline oxygen fraction 408, 428 delivered to the patient of about 1.5%. In the scenarios illustrated in FIGS. 4C and 4D, each of the waveforms 432, 442 indicates a change in oxygen fraction with respect to their respective baseline oxygen fraction 438, 448 delivered to the patient of about 3%. Based on the waveforms 402, 422, 432, 442 illustrated in FIGS. 4A to 4D, it can be determined that the patient 102 is spontaneously breathing with a patent airway.

The controller 138 may provide an indication to adjust the display associated with the user interface 140 to enlarge the waveform, or automatically adjust the display to enlarge the waveform when the flow rate meets or exceeds the patient's peak inspiratory demand. Typically, when the patient's peak inspiratory demand is met or exceeded, high flow rate gas flow is used, and any changes in the fraction of oxygen at the patient can be small, as illustrated in FIGS. 3A to 4D. Accordingly, it would be desirable to change the scale of the generated waveform such that the changes of fraction of oxygen monitored at the patient's airway can be more clearly visualised.

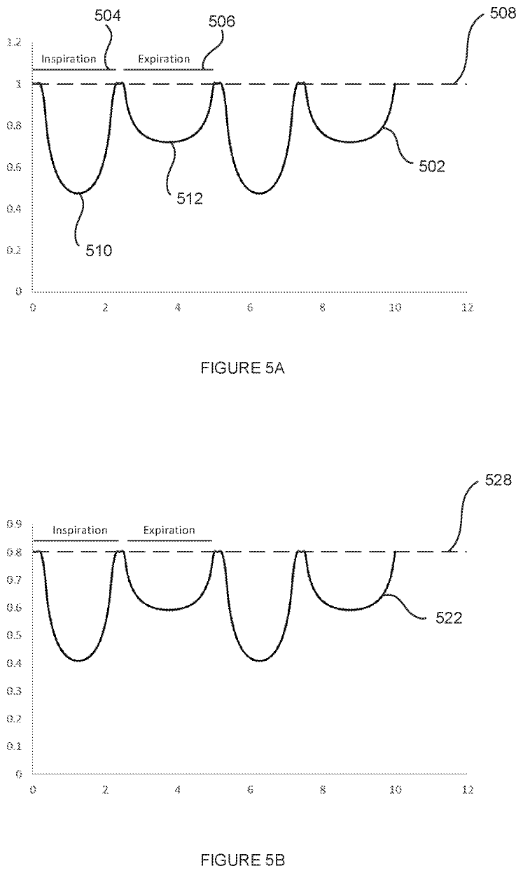

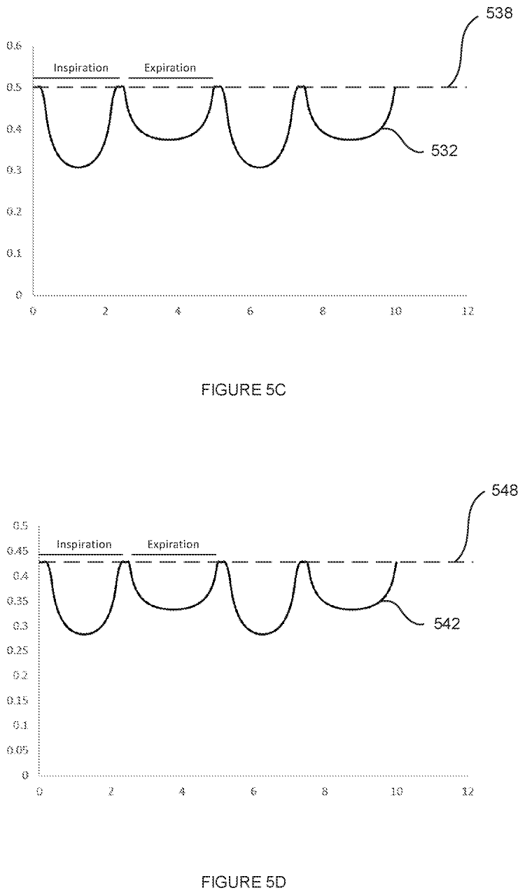

FIGS. 5A to 5D each illustrate waveforms 502, 522, 532, 542 representing a fraction of oxygen as measured inside the patient's 102 nose over two assumed respiratory cycles. The patient's mouth may be substantially closed. As more clearly shown in FIG. 5A, each respiratory cycle includes an inspiration phase 504 and expiration phase 506. The waveforms 502, 522, 532, 542 are generated in a scenario where the flow rate of the gas flow 106 is provided at a low flow rate, for example 10 LPM and the patient's peak inspiratory demand is determined to be roughly 30 LPM. In this scenario, the flow rate of the gas flow 106 does not meet the peak inspiratory demand of the patient 102.

The gas flow 106 provided by the system 100 in FIG. 5A is 100% oxygen (fraction of oxygen, FiO₂=1). Similar to FIGS. 2A, 3A and 4A, this is a scenario in which the baseline fraction of oxygen 508 delivered to the patient 102 is also 1 (FiO_{2(baseline)}=1). In some cases, the baseline fraction of oxygen is less than the fraction of oxygen provided by the system 100. For example, in FIGS. 5B to 5D, baseline fraction of oxygen 528, 538, 548 delivered to the patient 102 is roughly 0.8, 0.5, 0.43 respectively (FiO_{2(baseline)}=0.8, 0.5, 0.43).

Each of the waveforms 502, 522, 532, 542 indicate changes in the fraction of oxygen as presented by dips in the respective waveforms. For example, as illustrated in FIG. 5A, the waveform 502 indicates two dips 510, 512, each dip corresponding to an inspiration phase 504 or expiration phase 506 of an assumed respiratory cycle. Typically, when the patient's inspiratory demand is not met, the waveform 502 will indicate a reduction in the fraction of oxygen during each of inspiration and expiration phase 504, 506 of each respiratory cycle (also see FIGS. 2A to 2D). When the patient's inspiratory demand is not being met, the waveform 502 indicates two dips 510, 512 during a period of assumed respiratory cycle. Each dip 510, 512 in the waveform 502 represents a decrease in the fraction of oxygen at the patient 102 with respect to the baseline 508. Dip 510 in the waveform 502 is caused by entrainment during inspiration as the patient's inspiratory demand is not met. Dip 512 in the waveform 202 is caused by the patient's exhalation mixing with the delivered gas flow.

It has been observed that in the scenarios illustrated in FIGS. 5A and 5B, each of the waveforms 502, 522 indicates a change in oxygen fraction with respect to their respective baseline oxygen fraction 508, 528 delivered to the patient of about 53% and 49% respectively. In the scenarios illustrated in FIGS. 5C and 5D, each of the waveforms 532, 542 indicates a change in oxygen fraction with respect to their respective baseline oxygen fraction 538, 548 delivered to the patient of about 40% and 34% respectively. Based on the waveforms 502, 522, 532, 542 illustrated in FIGS. 5A to 5D, it can be determined that the patient 102 is spontaneously breathing with a patent airway.

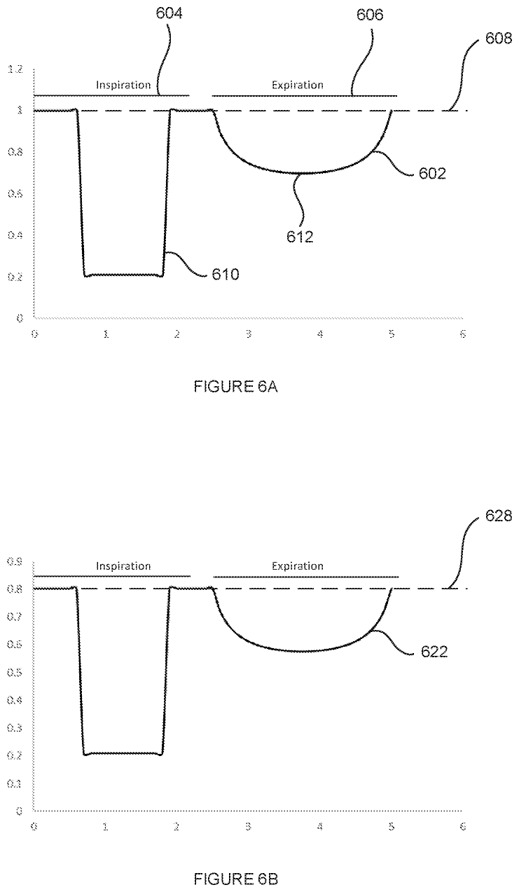

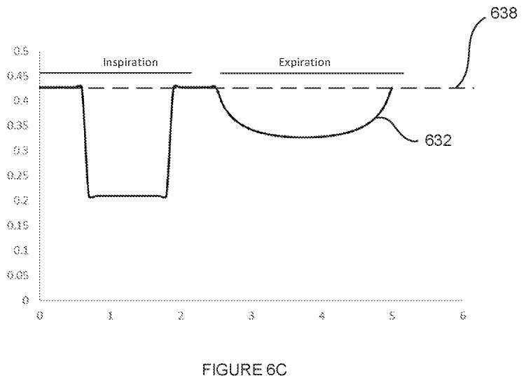

FIGS. 6A to 6C each illustrate waveforms 602, 622, 632 representing a fraction of oxygen as measured outside and proximate the patient's 102 mouth over one assumed respiratory cycle. As more clearly shown in FIG. 6A, the illustrated respiratory cycle includes an inspiration phase 604 and expiration phase 606. The waveforms 602, 622, 632 are generated in a scenario where the flow rate of the gas flow 106 is provided at a high flow rate, for example 30 LPM and the patient's peak inspiratory demand is determined to be roughly 40 LPM. In this scenario, the flow rate of the gas flow 106 does not meet the peak inspiratory demand of the patient 102.

The gas flow 106 provided by the system 100 in FIG. 6A is 100% oxygen (fraction of oxygen, FiO₂=1). In this scenario, the baseline fraction of oxygen 608 delivered to the patient 102 is also 1 (FiO_{2(baseline)}=1). In some cases, the baseline fraction of oxygen is less than the fraction of oxygen provided by the system 100. For example, in FIGS. 6B and 6C, baseline fraction of oxygen 628, 638 delivered to the patient 102 is roughly 0.8, 0.43 respectively (FiO_{2(baseline)}=0.8, 0.43). The gas flow 106 is delivered to the patient 102 via the nose.

Each of the waveforms 602, 622, 632 indicate changes in the fraction of oxygen as presented by dips in the respective waveforms. For example, as illustrated in FIG. 6A, the waveform 602 indicates two dips 610, 612, each dip corresponding to an inspiration phase 604 or expiration phase 606 of an assumed respiratory cycle. Typically, when the patient's peak inspiratory demand is not being met, the waveform 602 will indicate a reduction in the fraction of oxygen during each of inspiration and expiration phase 604, 606 of each respiratory cycle (also see FIGS. 2A to 2D, and FIGS. 5A to 5D). When the patient's peak inspiratory demand is not being met, the waveform 602 indicates two dips 610, 612 during a period of assumed respiratory cycle. Each dip 610, 612 in the waveform 602 represents a decrease in the fraction of oxygen at the patient 102 with respect to the baseline 608. As the patient's peak inspiratory demand is not being met, dip 610 in the waveform 602 illustrates that the patient 102 is breathing (entraining) in air via the mouth as the gas flow 106 delivered to the patient 102 via the nose is entirely delivered to the patient's lungs. Dip 612 in the waveform 602 is caused by the patient's exhaled breath mixing with the delivered gas flow (or a proportion thereof) and exiting the mouth.

It has been observed that in the scenarios illustrated in FIGS. 6A to 6C, each of the waveforms 602, 622, 632 indicates a maximum change in oxygen fraction with respect to their respective baseline oxygen fraction 608, 628, 638 delivered to the patient of about 79%, 74% and 51% respectively. Based on the waveforms 602, 622, 632 illustrated in FIGS. 6A to 6C, it can be determined that the patient 102 is spontaneously breathing with a patent airway.

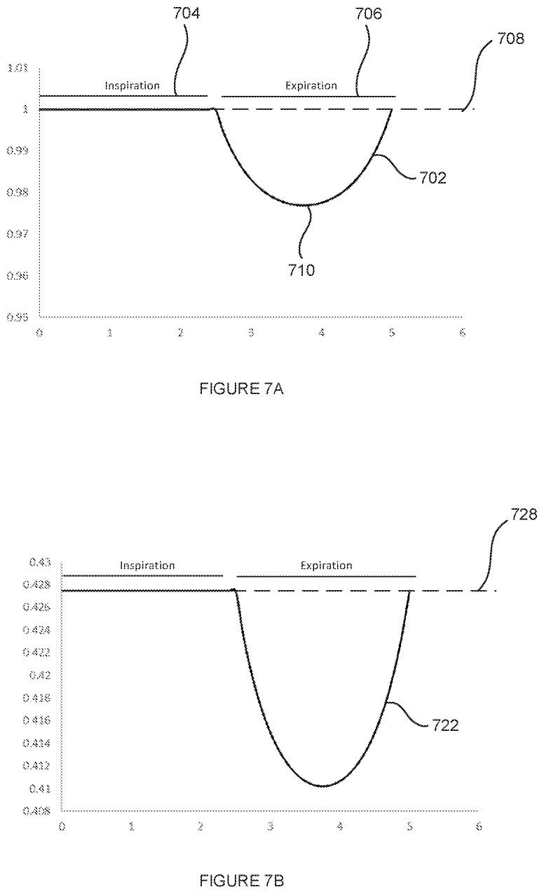

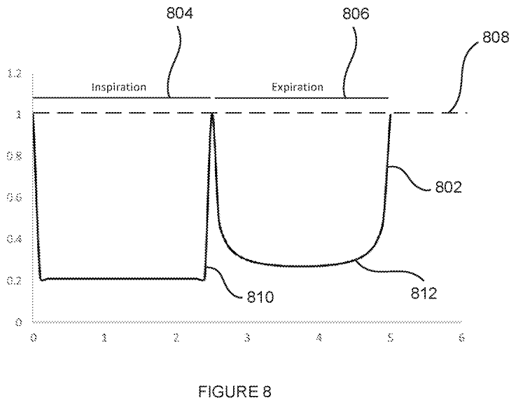

FIGS. 7A and 7B each illustrate waveforms 702, 722 representing a fraction of oxygen as measured outside and proximate the patient's 102 mouth over one assumed respiratory cycle. As more clearly shown in FIG. 7A, the illustrated respiratory cycle includes an inspiration phase 704 and expiration phase 706. The waveforms 702, 722 are generated in a scenario where the flow rate of the gas flow 106 is provided at a high flow rate, for example 70 LPM and the patient's peak inspiratory demand is determined to be roughly 30 LPM. In this scenario, the flow rate of the gas flow 106 meets or exceeds the peak inspiratory demand of the patient 102.

The gas flow 106 provided by the system 100 in FIG. 6A is 100% oxygen (fraction of oxygen, FiO₂=1). In this scenario, the baseline fraction of oxygen 708 delivered to the patient 102 is also 1 (FiO_{2(baseline)}=1). In some cases, the baseline fraction of oxygen is less than the fraction of oxygen provided by the system 100. For example, in FIG. 7B, baseline fraction of oxygen 728 at the patient 102 is roughly 0.427 (FiO_{2(baseline)}=0.427). The gas flow 106 is delivered to the patient 102 via the nose.

Each of the waveforms 702, 722 indicate changes in the fraction of oxygen as presented by dips in the respective waveforms. For example, as illustrated in FIG. 7A, the waveform 702 indicates one dip 710 corresponding to an expiration phase 706 of an assumed respiratory cycle. Typically, when the patient's peak inspiratory demand is being met or exceeded, the waveform 702 will indicate a reduction in the fraction of oxygen during the expiration phase 706 of each respiratory cycle. When the patient's peak inspiratory demand is being met or exceeded, the waveform 702 indicates one dip 710 during a period of assumed respiratory cycle. The dip 710 in the waveform 702 represents a decrease in the fraction of oxygen at the patient 102 with respect to the baseline 708. As the patient's peak inspiratory demand is being met or exceeded, the waveform 702 does not indicate any decrease in the fraction of oxygen measured outside/proximate the mouth corresponding to an inspiration phase of the patient's respiratory cycle. This is because excess gas flow delivered through the nose is flowing out via the patient's mouth. The measured fraction of oxygen outside/proximate the patient's mouth during an inspiration phase is therefore roughly equal to the fraction of oxygen delivered to the patient via the nose. Dip 710 in the waveform 702 is caused by the patient's exhaled breath mixing with the delivered gas flow (or a proportion thereof) and exiting the mouth.

In some embodiments, the patient is receiving a gas flow via the nose at a high flow rate that meets or exceeds the patient's inspiratory demand, and the sensor 136 is positioned inside, or outside and proximate the patient's mouth, and the patient is partially breathing through the mouth. In this scenario, the sensor 136 may detect a reduction in the fraction of oxygen during an inspiratory phase of the patient's respiratory cycle, corresponding to a dip in the waveform during an inspiratory phase of an assumed respiratory cycle. Accordingly, in some embodiments, when the patient's inspiratory demand is met or exceeded, the waveform may indicate two dips during a period of assumed respiratory cycle

It has been observed that in the scenarios illustrated in FIGS. 7A and 7B, each of the waveforms 702, 722 indicates a change in oxygen fraction with respect to their respective baseline oxygen fraction 708, 728 delivered to the patient of about 2.3% and 4% respectively. Based on the waveforms 702, 722 illustrated in FIGS. 7A and 7B, it can be determined that the patient 102 is spontaneously breathing with a patent airway.

FIG. 8 illustrates a waveform 802 representing a fraction of oxygen as measured outside and proximate the patient's 102 mouth over one assumed respiratory cycle. The illustrated respiratory cycle includes an inspiration phase 804 and expiration phase 806. The waveform 802 is generated in a scenario where the flow rate of the gas flow 106 is provided at a low flow rate, for example 2 LPM and the patient's peak inspiratory demand is determined to be roughly 30 LPM. In this scenario, the flow rate of the gas flow 106 does not meet the peak inspiratory demand of the patient 102.

The gas flow 106 provided by the system 100 in FIG. 6A is 100% oxygen (fraction of oxygen, FiO₂=1). In this scenario, the baseline fraction of oxygen 808 delivered to the patient 102 is also 1 (FiO_{2(baseline)}=1). In some cases, the baseline fraction of oxygen can be less than the fraction of oxygen provided by the system 100.

The waveform 802 indicates a change in the fraction of oxygen as presented by two dips 810, 812. Each dip 810, 812 corresponding to an inspiration phase 804 or expiration phase 806 of an assumed respiratory cycle. Typically, when the patient's peak inspiratory demand is not being met, the waveform 802 will indicate a reduction in the fraction of oxygen during each of inspiration and expiration phase 804, 806 of each respiratory cycle. When the patient's peak inspiratory demand is not being met, the waveform 802 indicates two dips 810, 812 during a period of assumed respiratory cycle. Each dip 810, 812 in the waveform 802 represents a decrease in the fraction of oxygen at the patient 102 with respect to the baseline 808. As the patient's peak inspiratory demand is not being met, dip 810 in the waveform 802 illustrates that the patient 102 is also breathing (entraining) in air via the mouth as the gas flow 106 delivered to the patient 102 via the nose is entirely delivered to the patient's lungs. Dip 812 in the waveform 802 is caused by the patient's exhaled breath mixing with the delivered gas flow (or a proportion thereof) and exiting the mouth.

It has been observed that in the scenario illustrated in FIG. 8, the waveform 802 indicates a change in oxygen fraction with respect to the baseline oxygen fraction 808 delivered to the patient of about 79%. Based on the waveform 802 illustrated in FIG. 8, it can be determined that the patient 102 is spontaneously breathing with a patent airway.

In FIGS. 2A to 2D, 5A to 5D, 6A to 6C, and 8, which illustrate scenarios when the patient's peak inspiratory demand is not being met, and the respective waveforms indicate two dips during a period of assumed respiratory cycle. In each of these figures, the dip corresponding to the inspiration phase (‘inspiration dip’) illustrates a greater reduction in the oxygen fraction than the dip corresponding to the expiration phase (‘expiration dip’). In some embodiments, the expiration dip may illustrate a greater reduction in the oxygen fraction than the inspiration dip. In some embodiments, the expiration dip may illustrate a similar reduction in the oxygen fraction as the inspiration dip.

Generally, for a given fraction of delivered oxygen, a relatively higher flow rate of gas flow delivered to the patient will correspond to smaller changes in the fraction of oxygen (in which each dip will have a smaller magnitude) as presented in the waveform to indicate spontaneous breathing. Conversely, for a given fraction of delivered oxygen, a relatively lower flow rate of gas flow delivered to the patient will correspond to larger changes in the fraction of oxygen (in which each dip will have a greater magnitude) as presented in the waveform to indicate spontaneous breathing.

Typically, to discount the effect of noise and/or other erroneous or interfering signals, the determination of spontaneous breathing with a patent airway may be based on at least one dip during a period of assumed respiratory cycle consistently over a plurality of respiratory cycles in the waveform. In particular, the determination of spontaneous breathing with a patent airway may be based on at least one dip during a period of assumed respiratory cycle consistently over a plurality of respiratory cycles in accordance with any one of the waveforms as described herein.

In particular, the waveform may consistently indicate at least one dip during a period of assumed respiratory cycle for at least 2 consecutive respiratory cycles, when the frequency of respiratory cycles is about 0.02 to 0.5 Hz (1 to 30 respiratory cycles per minute), or less than 0.5 Hz to enable determination that a patient is spontaneously breathing with a patent airway. Preferably, the waveform may consistently indicate at least one dip during a period of assumed respiratory cycle for 5 or more consecutive respiratory cycles. In some embodiments, the waveform may consistently indicate at least one dip during a period of assumed respiratory cycle for 2 to 5 consecutive respiratory cycles.

In some embodiments, the waveform may indicate at least one spike during a period of assumed respiratory cycle. The waveform may indicate at least one spike during a period of assumed respiratory cycle for at least 2 consecutive respiratory cycles. Each spike in the waveform represents an increase in the fraction of oxygen at the patient with respect to the baseline. Each spike may correspond with an expiration phase of the patient's respiratory cycle. Accordingly, the method may include determining that the patient is spontaneously breathing with a patent airway when the waveform indicates at least one spike during a period of assumed respiratory cycle. In relation to the system, the one or more controllers may be configured to determining that the patient is spontaneously breathing with a patent airway when the waveform indicates at least one spike during a period of assumed respiratory cycle.

Typically, the waveform may indicate one or more spikes following a reduction of the delivered oxygen fraction. Following a reduction of the delivered oxygen fraction, a reservoir of oxygen present in the patient's lungs can contribute to an increase in the fraction of oxygen at the patient with respect to the reduced baseline oxygen fraction during an expiration phase. The increase in the fraction of oxygen can be detected and represented in the waveform in the form of a spike.

In the event that a generated waveform indicates 0% or near 0% change in oxygen fraction with respect to their respective baseline oxygen fraction delivered to the patient, it may be determined that the patient is apnoeic or has an obstructed airway.

### Interpretation

This specification, including the claims, is intended to be interpreted as follows:

Embodiments or examples described in the specification are intended to be illustrative of the invention, without limiting the scope thereof. The invention is capable of being practised with various modifications and additions as will readily occur to those skilled in the art. Accordingly, it is to be understood that the scope of the invention is not to be limited to the exact construction and operation described or illustrated, but only by the following claims.

The mere disclosure of a method step or product element in the specification should not be construed as being essential to the invention claimed herein, except where it is either expressly stated to be so or expressly recited in a claim.

The invention may also be said broadly to consist in the parts, elements and features referred to or indicated in the specification of the application, individually or collectively, in any or all combinations of two or more of said parts, elements or features. Where, in the foregoing description reference has been made to integers or components having known equivalents thereof, those integers are herein incorporated as if individually set forth.

The terms in the claims have the broadest scope of meaning they would have been given by a person of ordinary skill in the art as of the relevant date.

The terms “a” and “an” mean “one or more”, unless expressly specified otherwise.

Neither the title nor the abstract of the present application is to be taken as limiting in any way as the scope of the claimed invention.

Where the preamble of a claim recites a purpose, benefit or possible use of the claimed invention, it does not limit the claimed invention to having only that purpose, benefit or possible use.

In the specification, including the claims, the term “comprise”, and variants of that term such as “comprises” or “comprising”, are used to mean “including but not limited to”, unless expressly specified otherwise, or unless in the context or usage an exclusive interpretation of the term is required.

The disclosure of any document referred to herein is incorporated by reference into this patent application as part of the present disclosure, but only for purposes of written description and enablement and should in no way be used to limit, define, or otherwise construe any term of the present application where the present application, without such incorporation by reference, would not have failed to provide an ascertainable meaning. Any incorporation by reference does not, in and of itself, constitute any endorsement or ratification of any statement, opinion or argument contained in any incorporated document. Moreover, unless specifically stated otherwise, reference to such external documents is not to be construed as an admission that such documents, or such sources of information, in any jurisdiction, are prior art, or form part of the common general knowledge in the art.

The claims defining the invention are as follows:

1. A method of monitoring oxygen at a patient to determine spontaneous breathing with a patent airway, the method including the steps of
providing a gas flow to a patient's airway, the gas flow including a predetermined fraction of oxygen,
monitoring the fraction of oxygen at the patient's airway,
generating a waveform representing the fraction of oxygen at the patient's airway, and
determining whether the patient is spontaneously breathing with a patent airway based on the waveform.

2. The method of claim 1, wherein when the patient is breathing with a patent airway, the waveform indicates changes in the fraction of oxygen for each respiratory cycle as measured at the patient's airway.

3. The method according to any one of the preceding claims, wherein the step of determining includes determining that the patient is spontaneously breathing with a patent airway when the waveform indicates a decrease in the fraction of oxygen with respect to a baseline fraction of oxygen delivered to the patient corresponding to an assumed respiratory cycle of the patient.

4. The method according to any one of the preceding claims, wherein the step of determining includes determining that the patient is apnoeic or has an obstructed airway when the waveform indicates insufficient change in the fraction of oxygen over a period of time as measured at the patient's airway.

5. The method of claim 4, including
automatically detecting that the patient is apnoeic or has an obstructed airway based on the waveform.

6. The method according to any one of the preceding claims, wherein the step of providing a gas flow to the patient's airway includes providing the gas flow through a non-sealing user interface.

7. The method of claim 6, wherein the non-sealing user interface includes a non-sealing nasal cannula.

8. The method according to any one of the preceding claims, wherein the step of determining includes determining that the patient is spontaneously breathing with a patent airway if the waveform indicates a change in oxygen fraction with respect to a baseline oxygen fraction delivered to the patient of more than about 10%.

9. The method according to any one of the preceding claims, wherein the step of determining includes determining that the patient is spontaneously breathing with a patent airway if the waveform indicates a change in oxygen fraction with respect to a baseline oxygen fraction delivered to the patient of more than about 30%.

10. The method according to any one of the preceding claims, further including determining that the patient is spontaneously breathing with a patent airway, if the waveform indicates at least two dips during a period of assumed respiratory cycle when an inspiratory demand of the patient is not met.

11. The method according to any one of claims 1 to 7, further including
determining that the patient is spontaneously breathing with a patent airway if
the waveform indicates at least two dips during a period of assumed respiratory cycle when an inspiratory demand of the patient is not met, and
the waveform indicates a change in oxygen fraction with respect to a baseline oxygen fraction delivered to the patient is about 10% to 30%.

12. The method according to any one of the preceding claims, wherein the step of providing a gas flow to the patient's airway includes providing the gas flow at a high flow rate, and
the step of determining includes determining that the patient is spontaneously breathing with a patent airway if the waveform indicates at least one dip during a period of assumed respiratory cycle.

13. The method according to any one of claims 10 to 12, wherein each dip in the waveform represents a decrease in the fraction of oxygen with respect to a baseline fraction of oxygen delivered to the patient.

14. The method according to any one of the preceding claims, wherein the step of providing a gas flow to a patient's airway includes providing the gas flow at a high flow rate of greater than 15 litres per minute (LPM).

15. A method according to any one of the preceding claims, wherein the step of providing a gas flow to a patient's airway includes providing the gas flow at a high flow rate of greater than 20 LPM.

16. A method according to any one of the preceding claims, wherein the step of providing a gas flow to a patient's airway includes providing the gas flow at a high flow rate of between about 20 LPM to 90 LPM.

17. A method according to any one of the preceding claims, wherein the step of providing a gas flow to a patient's airway includes providing the gas flow at a high flow rate of between about 40 LPM to 70 LPM.

18. A method according to any one of claims 1 to 11, wherein the step of providing a gas flow to the patient's airway includes providing the gas flow at a low flow rate, and
the step of determining includes determining that the patient is spontaneously breathing with a patent airway if the waveform indicates at least two dips during a period of assumed respiratory cycle.

19. The method according to any one of claims 1 to 11, wherein the step of providing a gas flow to a patient's airway includes providing the gas flow at a low flow rate of less than 20 LPM.

20. The method of claim 19, wherein the step of providing a gas flow to a patient's airway includes providing the gas flow at a low flow rate of less than 15 LPM.

21. The method according to any one of the preceding claims, further including upon determining that the patient apnoeic or has an obstructed airway based on the waveform, providing an indication that the patient is apnoeic or has an obstructed airway.

22. The method of claim 18, wherein the indication that the patient is apnoeic or has an obstructed airway includes any one or more of
a sound indicator,
a light indicator, and
a display message.

23. A method according to any one of the preceding claims, wherein the method is performed during a medical procedure when the patient has a diminished respiratory function or a risk of diminished respiratory function resulting from anaesthetic agents.

24. The method of claim 23, wherein the medical procedure is procedural sedation.

25. The method according to any one of the preceding claims, further including
setting the gas flow at a continuous flow rate independent of the patient's breathing, and
wherein the step of providing a gas flow to a patient's airway includes providing the gas flow at the continuous flow rate.

26. The method according to any one of the preceding claims, further including
automatically scaling a display of the waveform when the waveform indicates that a change in oxygen fraction with respect to a baseline oxygen fraction delivered to the patient is less than about 30%.

27. The method of claim 26, further including
automatically scaling a display of the waveform when the waveform indicates that a change in oxygen fraction with respect to a baseline oxygen fraction delivered to the patient is less than about 10%.

28. A method of claim 26 or 27, wherein the step of automatically scaling includes increasing the scale by a factor of 5 or more.

29. A method according to any one of the preceding claims, wherein the step of providing the gas flow to the patient's airway includes continuously providing the gas flow to the patient's airway, and
the step of monitoring includes continuously monitoring the fraction of oxygen at the patient's airway.

30. A system of monitoring oxygen at a patient to determine breathing or patent airway, the system including
a flow source for providing a gas flow to a patient's airway, the gas flow including a predetermined fraction of oxygen,
one or more sensors for monitoring the fraction of oxygen at the patient's airway, and
one or more controllers being configured to
generate a waveform representing the fraction of oxygen at the patient's airway based on input from the one or more sensors to allow for determination of whether the patient is spontaneously breathing with a patent airway.

31. The system of claim 30, wherein the one or more controllers is configured to determine whether the patient is spontaneously breathing with a patent airway.

32. The system of claim 30 or 31, wherein the one or more controllers is configured to determine that the patient is spontaneously breathing with a patent airway when the waveform indicates a decrease in the fraction of oxygen with respect to a baseline fraction of oxygen delivered to the patient corresponding to an assumed respiration cycle of the patient.

33. The system according to any one of claims 30 to 32, wherein the one or more controllers is configured to determine that the patient is apnoeic or has an obstructed airway when the waveform indicates insufficient change in the fraction of oxygen over a period of time as measured at the patient's airway.

34. The system according to any one of claims 30 to 33, further including a non-sealing patient interface for providing the gas flow to the patient's airway.

35. The system of claim 34, wherein the non-sealing patient interface includes a non-sealing nasal cannula.

36. The system according to any one of claims 30 to 35, further including a humidifier for humidifying the gas flow.

37. The system according to any one of claims 30 to 36, wherein the flow source includes a blower to facilitate movement of the gas flow.

38. The system according to any one of claims 30 to 36, wherein the flow source includes an oxygen source.

39. The system according to any one of claims 30 to 37, further including an inspiratory tube for providing the gas flow to the patient's airway, the inspiratory tube including a heating element for providing heat to the gas flow.

40. The system according to any one of claims 30 to 39, wherein the one or more controllers is configured to determine that the patient is spontaneously breathing with a patent airway if the waveform indicates a change in oxygen fraction with respect to a baseline oxygen fraction delivered to the patient of more than about 10%.

41. The system according to any one of claims 30 to 40, wherein the one or more controllers is configured to determine that the patient is spontaneously breathing with a patent airway if the waveform indicates a change in oxygen fraction with respect to a baseline oxygen fraction delivered to the patient of more than about 30%.

42. The system according to any one of claims 30 to 41, wherein the one or more controllers is further configured to determine that the patient is spontaneously breathing with a patent airway if the waveform indicates at least two dips during a period of assumed respiratory cycle when an inspiratory demand of the patient is not met.

43. The system according to any one of claims 30 to 42, wherein the one or more controllers is further configured to determine that the patient is spontaneously breathing with a patent airway if
the waveform indicates at least two dips during a period of assumed respiratory cycle when an inspiratory demand of the patient is not met, and
the waveform indicates a change in oxygen fraction with respect to a baseline oxygen fraction delivered to the patient is about 10% to 30%.

44. The system according to any one of claims 30 to 43, wherein
the flow source provides the gas flow to the patient's airway at a high flow rate, and
the one or more controllers is configured to determine that the patient is spontaneously breathing with a patent airway if the waveform indicates at least one dip during a period of assumed respiratory cycle.

45. The system according to any one of claims 30 to 44, wherein the flow source provides the gas flow to the patient's airway at a high flow rate of greater than 15 LPM.

46. The system according to any one of the claims 30 to 45, wherein the flow source provides the gas flow to the patient's airway at a high flow rate of greater than 20 LPM.

47. The system according to any one of the claims 30 to 46, wherein the flow source provides the gas flow to the patient's airway at a high flow rate of between about 20 LPM to 90 LPM.

48. The system according to any one of the claims 30 to 46, wherein the flow source provides the gas flow to the patient's airway at a high flow rate of between about 40 LPM to 70 LPM.

49. The system according to any one of claims 30 to 43, wherein the flow source provides the gas flow to the patient's airway at a low flow rate, and
the one or more controllers is configured to determine that the patient is spontaneously breathing with a patent airway if the waveform indicates at least two dips during a period of assumed respiratory cycle.

50. The system according to any one of claims 30 to 43, wherein the flow source provides the gas flow to the patient's airway at a low flow rate of less than 20 LPM.

51. The system of claim 50, wherein the flow source provides the gas flow to the patient's airway at a low flow rate of less than 15 LPM.

52. The system according to any one of claims 30 to 51, wherein the one or more controllers is further configured to provide an indication upon determining that the patient is apnoeic or has an obstructed airway based on the waveform.

53. The system of claim 52, wherein the indication that the patient is apnoeic or has an obstructed airway includes any one or more of
A sound indicator,
a light indicator, and
a display message.

54. The system according to any one of claims 30 to 53, wherein the system is a system of monitoring oxygen at a patient to determine spontaneous breathing with a patent airway during a medical procedure when the patient has a diminished respiratory function or a risk of diminished respiratory function resulting from anaesthetic agents.

55. The system of claim 54, wherein the medical procedure includes procedural sedation.

56. The system according to any one of claims 30 to 55, wherein the flow source provides a gas flow at a continuous flow rate independent of the patient's breathing.

57. The system according to any one of claims 30 to 56, further including
a display for displaying the waveform generated by the one or more controllers, and
wherein the one or more controllers is configured to automatically scale the display of the waveform when the waveform indicates that a change in oxygen fraction with respect to a baseline oxygen fraction delivered to the patient is less than about 30%.

58. The system of claim 57, wherein the one or more controllers is configured to automatically scale the display of the waveform when the waveform indicates that a change in oxygen fraction with respect to a baseline oxygen fraction delivered to the patient is less than about 10%.

59. The system according of claim 57 or 58, wherein the one or more controllers is configured to automatically scale the display of the waveform by a factor between 2 to 10.

60. The system according to any one of claims 30 to 59, wherein the one or more sensors includes an oxygen sensor.

61. The system according to any one of claims 30 to 60, wherein the flow source continuously provides the gas flow to the patient's airway, and
the one or more sensors continuously monitor the fraction of oxygen at the patient's airway.
